(19) United States
(12) Patent Application Publication
(10) Pub. No.: US 2016/0339010 A1
(43) Pub. Date: Nov. 24, 2016

(54) Treatment of Partly Controlled or Uncontrolled Severe Asthma

(71) Applicant: TAKEDA GMBH, Konstanz (DE)

(72) Inventors: Guido HANAUER, Konstanz (DE); Hermann TENOR, Konstanz (DE)

(73) Assignee: TAKEDA GMBH, Konstanz (DE)

(21) Appl. No.: 15/111,960

(22) PCT Filed: Jan. 20, 2015
(86) PCT No.: PCT/EP2015/050918

§ 371 Date: Jul. 15, 2016

Related U.S. Application Data
(60) Provisional application No. 61/930,111, filed on Jan. 22, 2014.

(51) Int. Cl. A61K 31/473; A61K 31/47

(52) U.S. Cl. CPC A61K 31/473; A61K 31/47

(57) ABSTRACT

Method for the treatment of partly controlled or uncontrolled severe asthma comprising administering either a) a phosphodiesterase 4 (PDE4) inhibitor or b) a phosphodiesterase 4 (PDE4) inhibitor and a leukotriene modifier as add-on to standard of care in severe asthma.

### FIELD OF THE INVENTION

The present invention is directed to therapies for the treatment of partly controlled or uncontrolled severe asthma. More particularly, the present invention is directed to the treatment of partly controlled or uncontrolled severe asthma with either a phosphodiesterase 4 inhibitor or a phosphodiesterase 4 inhibitor in combination with a leukotriene modifier.

### BACKGROUND OF THE INVENTION

Asthma is among the world's most prevalent diseases and is a serious global health problem. When not adequately controlled, asthma can severely limit daily life and is sometimes fatal. The prevalence of asthma is increasing in most countries, especially in children, with an estimated 300 million affected individuals. See, Global Strategy for Asthma Management and Prevention, Global Initiative for Asthma 2011, available at www.ginasthma.org. (hereafter “GINA 2011 Report”); Global Strategy for the Diagnosis and Management of Asthma in Children 5 years and younger, also available at www.ginasthma.org. (hereafter “GINA 2009 Children Report”); Masoli M, Fabian D, Holt S, Beasley R, Allergy 2004; 59: 469-478.

Asthma is a chronic inflammatory disorder of the airways in which many cells and cellular elements play a role. The chronic inflammation is associated with airway hyperresponsiveness that leads to recurrent episodes of wheezing, breathlessness, chest tightness, and coughing, particularly at night or in the early morning. These episodes are usually associated with widespread, but variable, airflow obstruction within the lung that is often reversible, either spontaneously or with treatment. GINA 2011 Report, Chapter 1, p. 2.

Asthma is a chronic disease of the lungs with inflammatory processes playing a central role in its pathogenesis. Barnes P J, Eur Respir Mon 2003; 23: 84-113. Infiltration of the airways by leukocytes with subsequent release of a range of inflammatory mediators causes microvascular leakage into the airways, mucus hypersecretion, bronchoconstriction and influx of further inflammatory cells into the airways. Asthma is identified by the presence of characteristic symptoms and functional abnormalities, whereby bronchoconstriction is a significant component of airway obstruction, but is reversible by bronchodilators.

The Global Strategy for Asthma Management and Prevention 2007 Report subdivided asthma by severity based on the level of symptoms, airflow limitation, and lung function variability into four categories: Intermittent, Mild Persistent, Moderate Persistent or Severe Persistent. Meanwhile, it has been recognized that asthma severity concerns both the severity of the underlying disease and its responsiveness to treatment. The main limitation of previous methods of classification of asthma severity was a poor ability to predict what treatment would be required and what a patient's response to that treatment might be. Taking into account these limitations, asthma severity is now (by consensus) classified on the basis of the intensity of treatment required to achieve good asthma control.

Asthma that can be well controlled with low intensity treatment, such as low-dose inhaled glucocorticosteroids, leukotriene modifiers or cromones, is regarded as mild asthma. Severe controllable asthma on the other hand, is asthma that requires high intensity treatment to maintain good control.

An important goal of asthma treatment is to achieve and maintain clinical control. According to the GINA 2011 Report and the GINA 2009 Children Report, the status of the current clinical control of a patient can be classified into three categories: controlled; partly controlled; and uncontrolled.

The levels of asthma control as classified in the GINA 2011 Report for patients older than 5 years can be described as follows:

The status “Controlled” asthma means all of the following criteria are satisfied: no (up to twice a week) daytime symptoms (cough, wheeze or dyspnea); no limitation of activities; no nocturnal symptoms/awakening; no need for reliever/rescue treatment (up to twice a week); and normal lung function (PEF—Peak Expiratory Flow Rate, or FEV₁—Forced Expiratory Volume in one second) without administration of a bronchodilator.

The status “Partly Controlled” asthma means that any of the following criteria is present: daytime symptoms (more than twice a week); any limitation of activities; any nocturnal symptoms/awakening; need for reliever/rescue treatment (more than twice a week); and lung function (PEF or FEV₁) of <80% predicted (without administration of bronchodilator).

Finally, “Uncontrolled” asthma is characterized by the presence of three or more of the features of Partly Controlled asthma.

Levels of asthma control as classified in the GINA 2009 Children Report for patients 5 years of age and younger are described as follows:

The status “Controlled” asthma means all of the following criteria are satisfied: no (up to twice a week) daytime symptoms (cough, wheeze or difficult breathing); no limitation of activities; no nocturnal symptoms/awakening; no need for reliever/rescue treatment (≦2 days/week).

The status “Partly Controlled” asthma means that any of the following criteria is present: daytime symptoms (more than twice a week); any limitation of activities; any nocturnal symptoms/awakening; need for reliever/rescue treatment (>2 days/week).

Finally, “Uncontrolled” asthma is characterized by the presence of three or more of the features of Partly Controlled asthma.

According to the GINA 2011 Report and GINA 2009 Children Report, the patient's current level of asthma control and current treatment determine the selection of pharmacologic treatment. If asthma is not controlled with a given treatment regimen, treatment should be stepped up until control is achieved. In case of Partly Controlled asthma, an increase in treatment should be considered, subject to whether more effective options are available (e.g. increased dose or an additional treatment).

GINA 2011 Report differentiates five treatment steps to achieve control in asthma patients older than 5 years. For a quick relief of symptoms, a reliever medication (rapid-onset bronchodilator, either short-acting or long-acting) should be provided at each treatment step.

GINA Step 1 (as needed reliever medication): Treatment with an as-needed reliever medication for usually untreated patients with occasional daytime symptoms (cough, wheeze, dyspnea occurring twice or less per week, or less frequently if nocturnal). A rapid-acting inhaled β2-agonist usually is the recommended reliever treatment for the majority of patients classified as GINA Step 1.

In GINA Step 2 to Step 5 treatments, the as-needed reliever treatment is combined with a regular controller treatment.

GINA Step 2 (reliever medication plus a single controller): To the treatment with the as-needed reliever, a low dose inhaled glucocorticosteroid (hereafter “ICS”) is added as the initial controller treatment. Alternative controller medications may include leukotriene modifiers, and less preferably, sustained release theophylline or cromones.

GINA Step 3 (reliever medication plus one or two controllers): In Step 3 it is recommended to combine a low-dose of inhaled glucocorticosteroid with an inhaled long-acting β2-agonist. Alternatively, one may increase the dose of the inhaled glucocorticosteroid from a low to a medium dose. Another alternative treatment option in Step 3 is the combination of a low dose inhaled glucocorticosteroid with a leukotriene modifier.

GINA Step 4 (reliever medication plus two or more controllers): The selection of treatment at step 4 should be based, if possible, on prior treatment selections at Steps 2 and 3. The combination of a medium- or high-dose inhaled glucocorticosteroid with an inhaled long-acting β2-agonist (hereafter “LABA”) is the usually preferred treatment at Step 4. Leukotriene modifiers or low-dose sustained-release theophylline may additionally be added to the combination of medium- or high-dose inhaled glucocorticosteroid and long-acting β2-agonists.

Gina Step 5 (reliever medication plus additional controller options): In Step 5, additional controllers may be added to the already applied Step 4 medication. Additional controllers may be low dose oral glucocorticosteroids or anti-IgE treatment.

The GINA 2009 Children Report does not contain a corresponding Step 1 to Step 5 treatment system for children younger than 5 years. When asthma control is not achieved in this group of patients by treatment with rapid-acting (inhaled) β2-agonists on an as needed basis, additionally a regular treatment with a low dose inhaled glucocorticosteroid is recommended. When asthma control is not achieved even by this treatment, doubling the initial dose of inhaled glucocorticosteroid is the recommended next treatment option. The best treatment for children younger than 5 years, whose asthma is not controlled on twice the initial dose of inhaled glucocorticosteroid, has not been established. According to the GINA 2009 Children Report, options to improve the control of the child's asthma in such a situation include further increasing the dose of the inhaled glucocorticosteroid, or adding a leukotriene modifier, theophylline, or a low dose of oral glucocorticosteroids for a few weeks.

Inhaled glucocorticosteroids (ICS) and long-acting β₂ agonists (LABAs) currently form the mainstay of maintenance treatment for the majority of asthma patients older than 5 years. International guidelines, such as those issued in the 2011 GINA Report advocate the combined use of ICS/LABA as maintenance therapy for patients with asthma older than 5 years who remain symptomatic despite low doses of ICS.

However, approximately 10% of all patients suffer from uncontrolled asthma, as defined by the GINA guidelines, even with stepped treatment through Step 5, including treatment with high-dose ICS and LABA. Wenzel S., Am J Respir Crit Care Med 2005; 172: 149-60. ICS in particular, may suppress airway inflammation and reduce airway hyper-responsiveness to temporarily control and prevent asthma symptoms, but they are not without limitations and the risk of systemic adverse effects increases with dose and with parenteral administration, limiting their prolonged use. In addition, many of the severe asthma patients, in particular those with a higher percentage of neutrophils in sputum, just do not respond to ICS or oral administered glucocorticosteroids.

Another approach to the treatment of asthma involves the physiological regulation of pro-inflammatory and immune cell function, which is mediated via intracellular cyclic adenosine monophosphate (cAMP). Phosphodiesterases (PDE) found in inflammatory cells, and in particular PDE4, mediate the breakdown of cAMP. Inhibition of PDE4 leads to an increase of intracellular cAMP levels, which in turn suppresses the inflammatory response. Chung K F., Eur J Pharmacol 2006; 533:110-117. Up to now, no PDE-4 inhibitor has been approved for use in the treatment of asthma.

From a small, open-label Investigator initiated trial there was reported a significantly improved FEV₁, morning PEF and Forced vital capacity (FVC), and reduced accumulation of neutrophils in induced sputum with Roflumilast, a selective PDE4 inhibitor (on top of standard medication of Fluticasone 500 mcg/Salmeterol 50 mcg twice daily), in patients with severe and uncontrolled bronchial asthma. Sadigov, A S, Am J. Respir. Crit. Care Med. 183; 2011:A4501.

Cysteinyl leukotrienes mediate tissue edema, infiltration and activation of inflammatory cells. They are potent inflammatory mediators, produced by the 5-lipoxygenase pathway of arachidonic acid metabolism, and are believed to play a role in the pathophysiology of asthma by mediating bronchoconstriction and inflammatory reactions throughout different cells.

The role of leukotriene receptor antagonists (LTRAs) in asthma treatment, and in particular, in not adequately controlled asthma treatment is under debate.

Clinical studies have demonstrated that leukotriene modifiers have a small and variable bronchodilator effect, reduce symptoms including cough, improve lung function and asthma exacerbations. Dicpinigaitis P V et al, J Asthma 2002; 39(4) 291-297. Leukotriene modifiers may be also used as an alternative treatment for adult patients with mild persistent asthma (Noonan M J et al Eur Respir J 1998, 11, 1232-1239) and some patients with aspirin sensitive asthma respond well to leukotriene modifiers. Dahlen et al, Am J Respir Crit Care Med 1998, 157, 1187-1194.

However, when used alone as a controller, the effects of leukotriene modifiers are generally less than that of low doses of inhaled glucocorticosteroids, and, in patients already on inhaled glucocorticosteroids leukotriene modifiers cannot substitute for this treatment without risking loss of asthma control. Bleecker E R et a, J Allergy Clin Immunol 2000, 105, 1123-1129.

In addition, while leukotriene modifiers are mentioned in the GINA 2011 Report as an alternative controller medication to low dose inhaled gluco-corticosteroids for GINA Step 2 patients, it is indicated at the same time that this alternative is particularly appropriate for patients who are unable or unwilling to use inhaled glucocorticosteroids or who experience intolerable side effects such as persistent hoarseness from inhaled glucocorticosteroid treatment. Leukotriene modifiers are also indicated in the GINA 2011 Report as an add-on to medium dose of inhaled glucocorticosteroids in patients on GINA Step 2 treatment, but here too, the recommended treatment option for children 5 years of age and older, adolescents and adults is the addition of an inhaled long-acting β2-agonist to the medium dose of inhaled glucocorticosteroids. In addition, leukotriene modifiers have also been tested as an add-on to medium or high-dose inhaled glucocorticosteroids in patients classified as GINA Step 3, but here too the achieved benefit usually was less than that achieved with the addition of long-acting β2-agonists. Laviolette et al Am J Respir Crit Care Med 1999, 160, 1862-1868.

Furthermore, a study by the American Lung Association into add-on therapy to inhaled glucocorticosteroids with leukotriene modifiers and theophylline reported no benefit of either therapy. The American lung Association Asthma Clinical Research Centres Am J Respir Crit Care Med 2007, 175, 235-242. Finally, the failure of montelukast to reduce sputum eosinophilia in patients with high-dose corticosteroid dependent asthma has been reported by L. Jayaram et al in Eur Respir J 2005, 25, 41-46.

International patent applications WO2009/052624 (U.S. patent application No. 2010-0210611), WO2003/024488 and WO02/38155 (U.S. Pat. No. 6,528,527) disclose the treatment of bronchial and respiratory disorders with a combination of a PDE4 inhibitor (including, for example roflumilast) and a leukotriene antagonist (including montelukast).

Although most asthma cases can be satisfactorily controlled with glucocorticosteroids or a combination of a glucocorticosteroid and a bronchodilator, there remains a significant proportion of asthma patients (approximately 10%) of all ages and both genders who continue to be symptomatic and at risk of exacerbations and not adequately controlled severe asthma, despite the established administration of high-dose ICS or other controllers, such as LABAs. Wenzel S., Am J Respir Crit Care Med 2005; 172:149-60. This sub-population of asthma patients as defined per GINA steps 4 and 5 has the greatest morbidity and need for health care utilization and costs. Thus, there is still a high unmet medical need for new pharmacotherapies to treat patients with partly controlled or uncontrolled severe asthma, owing to the lack of efficacious anti-inflammatory agents other than steroids. Therefore, alternative therapies that can be used with either ICS or ICS/LABA combinations are needed.

### DEFINITIONS

As used herein, the phrase “therapeutically effective amount” refers to the amount of active compound or pharmaceutical agent, or in the case of combination therapy, the combined amount of each compound or pharmaceutical agent, that elicits the biological or medicinal response that is being sought in a tissue, system, animal, individual or human by a researcher, veterinarian, medical doctor or other clinician, which includes one or more of the following:
(1) inhibiting the disease and its progression; for example, inhibiting a disease, condition or disorder in an individual who is experiencing or displaying the pathology or symptomatology of the disease, condition or disorder (i.e., arresting further development of the pathology and/or symptomatology) such as in the case of partly controlled or uncontrolled severe asthma, inhibiting the chronic inflammation associated with airway hyperresponsiveness that leads to recurrent episodes of wheezing, breathlessness, chest tightness, and coughing, usually associated with widespread, but variable airflow obstruction within the lung; and
(2) ameliorating the disease; for example, ameliorating a disease, condition or disorder in an individual who is experiencing or displaying the pathology or symptomatology of the disease, condition or disorder (i.e., reversing the pathology and/or symptomatology) such as in the case of partly controlled or uncontrolled severe asthma, decreasing the chronic inflammation associated with airway hyperresponsiveness that leads to recurrent episodes of wheezing, breathlessness, chest tightness, and coughing, usually associated with widespread, but variable airflow obstruction within the lung.

As used herein, “patient” refers to adult patients, adolescent patients 15 years of age and older, pediatric patients older than 5 and younger than 15 years of age and pediatric patients 5 years of age and younger. “Patient” without any further explanation includes all above mentioned patient groups.

As used herein, the meaning of the phrase “patient with partly controlled severe asthma” depends on the age of the patient to be treated:

For adult patients and adolescent patients 15 years of age and older, the phrase means that the adult patient or adolescent patient 15 years of age and older, suffers from at least one symptom selected from the group consisting of (i) daytime cough, wheeze or dyspnea, experienced more than twice a week; (ii) any limitation of daily activities; (iii) nocturnal cough, wheeze, dyspnea or asthma-related awakening; (iv) need for reliever/rescue treatment more than twice a week; and (v) lung function (PEF or FEV₁) of less than 80% of the predicted value (without administration of a bronchodilator), despite treatment with a rapid-acting β2-agonist on an as needed basis and maintenance treatment with medium or high dose inhaled glucocorticosteroid (ICS) plus a long acting β2-agonist. In this particular patient group, a medium dose inhaled glucocorticosteroid means a daily dose of, for example >500-1000 microgram (hereinafter “mcg”) beclomethasone dipropionate (CFC), >250-500 mcg beclomethasone dipropionate (HFA), ≧400-800 mcg budesonide, >160-320 mcg ciclesonide, >1000-2000 mcg flunisolide, >250-500 mcg fluticasone propionate, ≧400 mcg mometasone furoate or >1000-2000 mcg triamcinolone acetonide. Further, in this particular patient group, a high dose inhaled glucocorticosteroid means a daily dose of, for example >1000-2000 mcg beclomethasone dipropionate (CFC), >500-1000 mcg beclomethasone dipropionate (HFA), >800-1600 mcg budesonide, >320-1280 mcg ciclesonide, >2000 mcg flunisolide, >500-1000 mcg fluticasone propionate, 800 mcg mometasone furoate or >2000 mcg triamcinolone acetonide.

For pediatric patients older than 5 and younger than 15 years of age, the phrase means that the pediatric patient older than 5 and younger than 15 years of age suffers from at least one symptom selected from the group consisting of (i) daytime cough, wheeze or dyspnea, experienced more than twice a week; (ii) any limitation of daily activities; (iii) nocturnal cough, wheeze, dyspnea or asthma related awakening; (iv) need for reliever/rescue treatment more than twice a week; and (v) lung function (PEF or FEV₁) of less than 80% of the predicted value (without administration of a bronchodilator), despite treatment with a rapid-acting β2-agonist on an as needed basis and maintenance treatment with medium or high dose inhaled glucocorticosteroid (ICS) plus a long acting β2-agonist. In this particular patient group, a medium dose inhaled glucocorticosteroid means a daily dose of, for example >200-400 mcg beclomethasone dipropionate, >200-400 mcg budesonide, >500-1000 mcg budesonide nebulized, >160-320 mcg ciclesonide, >750-1250 mcg flunisolide, >200-500 mcg fluticasone propionate, ≧200 mcg mometasone furoate or >800-1200 mcg triamcinolone acetonide. Further, in this particular patient group, a high dose inhaled glucocorticosteroid means a daily dose of for example >400 mcg beclomethasone dipropionate, >400 mcg budesonide, >1000 mcg budesonide nebulized, >320 mcg ciclesonide, >1250 mcg flunisolide, >500 mcg fluticasone propionate, ≧400 mcg mometasone furoate or >1200 mcg triamcinolone acetonide.

For pediatric patients younger than 5 years of age, the phrase means that the pediatric patient younger than 5 years of age suffers from at least one symptom selected from the group consisting of (i) daytime wheezing, cough or difficult breathing experienced more than twice a week; (ii) any limitation of daily activities; (iii) nocturnal cough, wheezing, difficult breathing or asthma related awakening; and (iv) need for reliever/rescue treatment more than 2 days a week, despite treatment with a rapid-acting β2-agonist on an as needed basis and maintenance treatment with a double low dose inhaled glucocorticosteroid (ICS). In this particular patient group, double low dose inhaled glucocorticosteroid means a daily dose of, for example 200 mcg beclomethasone dipropionate, 400 mcg budesonide (MDI+spacer), 1000 mcg budesonide nebulized or 200 mcg fluticasone propionate.

As used herein, the meaning of the phrase “patient with uncontrolled severe asthma” depends on the age of the patient to be treated:

For adult patients and adolescent patients 15 years of age and older, the phrase means that the adult patient or adolescent patient 15 years of age and older, suffers from at least three symptom(s) selected from the group consisting of (i) daytime cough, wheeze or dyspnea, experienced more than twice a week; (ii) any limitation of daily activities; (iii) nocturnal cough, wheeze, dyspnea or asthma related awakening; (iv) need for reliever/rescue treatment more than twice a week; and (v) lung function (PEF or FEV₁) of less than 80% of the predicted value (without administration of a bronchodilator), despite treatment with a rapid-acting β2-agonist on an as needed basis and maintenance treatment with medium or high dose inhaled glucocorticosteroid (ICS) plus a long acting β2-agonist. In this particular patient group, a medium dose inhaled glucocorticosteroid means a daily dose of, for example >500-1000 mcg beclomethasone dipropionate (CFC), >250-500 mcg beclomethasone dipropionate (HFA) >250-500 mcg, ≧400-800 mcg budesonide, >160-320 mcg ciclesonide, >1000-2000 mcg flunisolide, >250-500 mcg fluticasone propionate, 400 mcg mometasone furoate or >1000-2000 mcg triamcinolone acetonide. Further, in this particular patient group, a high dose inhaled glucocorticosteroid means a daily dose of, for example >1000-2000 mcg beclomethasone dipropionate, >800-1600 mcg budesonide, >320-1280 mcg ciclesonide, >2000 mcg flunisolide, >500-1000 mcg fluticasone propionate, ≧800 mcg mometasone furoate or >2000 mcg triamcinolone acetonide.

For pediatric patients older than 5 and younger than 15 years of age, the phrase means that the pediatric patient older than 5 and younger than 15 years of age suffers from at least three symptoms selected from the group consisting of (i) daytime cough, wheeze or dyspnea, experienced more than twice a week; (ii) any limitation of daily activities; (iii) nocturnal cough, wheeze, dyspnea or asthma related awakening; (iv) need for reliever/rescue treatment more than twice a week; and (v) lung function (PEF or FEV₁) of less than 80% of the predicted value (without administration of a bronchodilator), despite treatment with a rapid-acting β2-agonist on an as needed basis and maintenance treatment with medium or high dose inhaled glucocorticosteroid (ICS) plus a long acting β2-agonist. In this particular patient group, a medium dose inhaled glucocorticosteroid means a daily dose of, for example >200-400 mcg beclomethasone dipropionate, >200-400 mcg budesonide, >500-1000 mcg budesonide nebulized, >160-320 mcg ciclesonide, >750-1250 mcg flunisolide, >200-500 mcg fluticasone propionate, ≧200 mcg mometasone furoate or >800-1200 mcg triamcinolone acetonide. Further, in this particular patient group, a high dose inhaled glucocorticosteroid means a daily dose of, for example >400 mcg beclomethasone dipropionate, >400 mcg budesonide, >1000 mcg budesonide nebulized, >320 mcg ciclesonide, >1250 mcg flunisolide, >500 mcg fluticasone propionate, ≧400 mcg mometasone furoate or >1200 mcg triamcinolone acetonide.

For pediatric patients younger than 5 years of age, the phrase means that the pediatric patient younger than 5 years of age suffers from at least three symptoms selected from the group consisting of (i) daytime wheezing, cough or difficult breathing experienced more than twice a week; (ii) any limitation of daily activities; (iii) nocturnal cough, wheezing, difficult breathing or asthma related awakening; and (iv) need for reliever/rescue treatment more than 2 days a week; despite treatment with a rapid-acting β2-agonist on an as needed basis and maintenance treatment with a double low dose inhaled glucocorticosteroid (ICS). In this particular patient group, a double low dose inhaled glucocorticosteroid means a daily dose of, for example 200 mcg beclomethasone dipropionate, 400 mcg budesonide (MDI+spacer), 1000 mcg budesonide nebulized or 200 mcg fluticasone propionate.

The phosphodiesterase 4 (PDE4) inhibitor used in this invention has the chemical name “5-((2R,4aR,10bR)-9-ethoxy-2-hydroxy-8-methoxy-1,2,3,4,4a,10b-hexahydro-phenanthridin-6-yl)-1-methylpyridin-2(1H)-one” and is hereinafter referred to as “Compound A”.

“Concurrent administration”, as used herein, means that both the PDE4 inhibitor (in particular Compound A or a pharmaceutically acceptable salt thereof) and the leukotriene modifier (in particular montelukast or a pharmaceutically acceptable salt thereof) (a) are administered to a patient in need of the treatment in a single dosage form for simultaneous, concomitant administration or (b) are administered to a patient in need of the treatment in two separate dosage forms, wherein one dosage form contains the PDE4 inhibitor and the other dosage form contains the leukotriene modifier, and the two separate dosage forms are administered immediately one after the other. In this context, the two separate dosage forms are administered immediately one after the other, if the two dosage forms are administered within between 0 and 15 minutes of each other; or more preferably within between 0 and 5 minutes of each other; or most preferably within between 0 and 1 minute of each other.

“Sequential administration”, as used herein, means that the PDE4 inhibitor (in particular Compound A or a pharmaceutically acceptable salt thereof) are administered to the patient in need of the treatment in one dosage form and the leukotriene modifier (in particular montelukast or a pharmaceutically acceptable salt thereof) is administered to the patient in need of the treatment in another separate dosage form, wherein the second dosage form is administered to the patient in need of the treatment while the first dosage form still has an effect on the patient being treated. In a preferred embodiment of the invention the first and the second dosage form are administered in such a time interval that the effect of the combined treatment on the patient being treated is a synergistic effect. In this context, the two separate dosage forms are administered sequentially, if the two dosage forms are administered more than 15 minutes apart.

“Asthma Exacerbations”, as used herein, are recognized as any episodes of worsening of asthma that are troublesome to patients, and that prompt a need for a change in treatment. A severe asthma exacerbation is defined by the need for oral or parenteral glucocorticosteroid intake for at least 3 days, and/or an emergency room visit or in-patient hospitalization requiring use of systemic corticosteroids for the treatment of asthma. A moderate asthma exacerbation is an event that, when recognized, results in a temporary change in the treatment to prevent the exacerbation from becoming severe. It is defined as an event that includes either a deterioration in symptoms, deterioration in lung function, or increased reliever use. These symptoms should persist for at least 2 days or more, but will not be severe enough to warrant systemic corticosteroid use and/or hospitalization.

“Limitation of (daily) activities”, as used herein, relates in connection with pediatric patients 5 years of age and younger to a limitation of activities, such as, for example, exercise, vigorous play or laughing due to the underlying disease (i.e. due to coughing, wheezing, or having difficulty breathing).

“Limitation of activities”, as used herein, relates in connection with patients older than 5 years to a limitation of typical age-dependent activities, due to the underlying disease.

### SUMMARY OF THE INVENTION

The present invention is directed to a method of treating partly controlled or uncontrolled severe asthma, not adequately controlled despite treatment according to GINA Step 4. The method includes the step of administering to a patient suffering from partly controlled or uncontrolled severe asthma, not adequately controlled despite treatment according to GINA Step 4, a therapeutically effective amount of a) a phosphodiesterase 4 (PDE4) inhibitor or b) a phosphodiesterase 4 (PDE4) inhibitor and a leukotriene modifier.

In another embodiment, the present invention is directed to a method of treating partly controlled or uncontrolled severe asthma, not adequately controlled despite treatment with a medium dose inhaled glucocorticosteroid (ICS) plus a long-acting β2-agonist. The method includes the step of administering to a patient suffering from partly controlled or uncontrolled severe asthma, not adequately controlled despite treatment with a medium dose inhaled glucocorticosteroid (ICS) plus a long-acting β2-agonist, a therapeutically effective amount of a) a phosphodiesterase 4 (PDE4) inhibitor or b) a phosphodiesterase 4 (PDE4) inhibitor and a leukotriene modifier.

In another embodiment, the present invention is directed to a method of treating partly controlled or uncontrolled severe asthma, not adequately controlled despite treatment with a rapid-acting β2-agonist on an as-needed basis and maintenance treatment with a medium dose inhaled glucocorticosteroid (ICS) plus a long-acting β2-agonist. The method includes the step of administering to a patient suffering from partly controlled or uncontrolled severe asthma, not adequately controlled despite treatment with a rapid-acting β2-agonist on an as-needed basis and maintenance treatment with a medium dose inhaled glucocorticosteroid (ICS) plus a long-acting β2-agonist, a therapeutically effective amount of a) a phosphodiesterase 4 (PDE4) inhibitor or b) a phosphodiesterase 4 (PDE4) inhibitor and a leukotriene modifier.

In yet another embodiment, the present invention is directed to a method of treating partly controlled or uncontrolled severe asthma, not adequately controlled despite treatment with a high dose inhaled glucocorticosteroid (ICS) plus a long-acting β2-agonist. The method includes the step of administering to a patient suffering from partly controlled or uncontrolled severe asthma, not adequately controlled despite treatment with a high dose inhaled glucocorticosteroid (ICS) plus a long-acting β2-agonist, a therapeutically effective amount of a) a phosphodiesterase 4 (PDE4) inhibitor or b) a phosphodiesterase 4 (PDE4) inhibitor and a leukotriene modifier.

In another embodiment, the present invention is directed to a method of treating partly controlled or uncontrolled severe asthma, not adequately controlled despite treatment with a rapid-acting β2-agonist on an as-needed basis and maintenance treatment with a high dose inhaled glucocorticosteroid (ICS) plus a long-acting β2-agonist. The method includes the step of administering to a patient suffering from partly controlled or uncontrolled severe asthma, not adequately controlled despite treatment with a rapid-acting β2-agonist on an as-needed basis and maintenance treatment with a high dose inhaled glucocorticosteroid (ICS) plus a long-acting β2-agonist, a therapeutically effective amount of a) a phosphodiesterase 4 (PDE4) inhibitor or b) a phosphodiesterase 4 (PDE4) inhibitor and a leukotriene modifier.

In another embodiment, the present invention is directed to a method of treating partly controlled or uncontrolled severe asthma, not adequately controlled despite treatment with double low dose inhaled glucocorticosteroid (ICS). The method includes the step of administering to a pediatric patient 5 years of age and younger suffering from partly controlled or uncontrolled severe asthma, not adequately controlled despite treatment with double low dose inhaled glucocorticosteroid (ICS), a therapeutically effective amount of a) a phosphodiesterase 4 (PDE4) inhibitor or b) a phosphodiesterase 4 (PDE4) inhibitor and a leukotriene modifier.

In yet another embodiment, the present invention is directed to a method of treating partly controlled or uncontrolled severe asthma, not adequately controlled despite treatment with a rapid-acting β2-agonist on an as-needed basis and maintenance treatment with a double low dose inhaled glucocorticosteroid (ICS). The method includes the step of administering to a pediatric patient 5 years of age and younger suffering from partly controlled or uncontrolled severe asthma, not adequately controlled despite treatment with a rapid-acting β2-agonist on an as-needed basis and maintenance treatment with a double low dose inhaled glucocorticosteroid (ICS), a therapeutically effective amount of a) a phosphodiesterase 4 (PDE4) inhibitor or b) a phosphodiesterase 4 (PDE4) inhibitor and a leukotriene modifier.

### DETAILED DESCRIPTION OF THE INVENTION

The present invention provides, inter alia, compositions and methods for the treatment of partly controlled or uncontrolled severe asthma. In particular, the invention is directed to the treatment of partly controlled or uncontrolled severe asthma in adult patients, adolescent patients 15 years of age and older and pediatric patients older than 5 and younger than 15 years of age, not adequately controlled despite treatment according to GINA Step 4 (from the GINA 2011 Report), or more particularly not adequately controlled despite treatment with a rapid-acting β2-agonist on an as needed basis and maintenance treatment with medium or high dose inhaled glucocorticosteroid (ICS) plus a long acting β2-agonist. The invention is furthermore directed to the treatment of partly controlled or uncontrolled severe asthma in pediatric patients 5 years of age and younger, not adequately controlled despite treatment with a rapid-acting β2-agonist on an as needed basis and maintenance treatment with double low dose inhaled glucocorticosteroid (ICS). Such treatment is provided by administering to these patient groups suffering from partly controlled or uncontrolled severe asthma, a therapeutically effective amount of a) a phosphodiesterase 4 (PDE4) inhibitor or b) the combination of a phosphodiesterase 4 (PDE4) inhibitor and a leukotriene modifier.

In case of co-administration of the phosphodiesterase 4 (PDE4) inhibitor and the leukotriene modifier (e.g., concurrent or sequential administration) these two agents provide a synergistic effect in the treatment of partly controlled or uncontrolled severe asthma, compared to the sum of effects seen with administration of these two agents alone. The synergistic effect seen by such co-administration permits the dosage of one or both of these agents to be reduced while still obtaining the same clinical effect, thereby reducing the incidence and/or severity of side effects seen with the administration of one or both of these compounds.

In case of co-administration of the phosphodiesterase 4 (PDE4) inhibitor and the leukotriene modifier, it is believed that by taking advantage of both mechanisms of action of these two classes of compounds a even more efficacious treatment of partly controlled or uncontrolled severe asthma can be achieved.

PDE4 Inhibitors

“Compound A”

The chemical name of Compound A is 5-((2R,4aR,10bR)-9-ethoxy-2-hydroxy-8-methoxy-1,2,3,4,4a,10b-hexahydro-phenanthridin-6-yl)-1-methylpyridin-2(1H)-one. Its empirical formula is C₂₂H₂₆N₂O₄ and the molecular weight is 382.4.

The phosphodiesterase-4 inhibitor Compound A is disclosed in U.S. Pat. No. 8,324,391 (hereby incorporated by reference in its entirety), and has the following chemical structure:

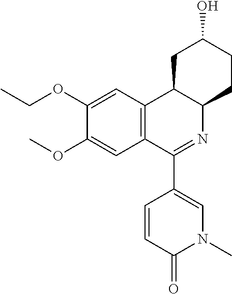 [SMILES: CCOc1cc2c(cc1OC)C(c1ccc(=O)n(C)c1)=N[C@@H]1CC[C@@H](O)C[C@H]21]

As pharmaceutically acceptable salts of Compound A may be mentioned the hydrochloride, the fumarate and L-tartrate salt of Compound A. Salts of Compound A are, for example, disclosed in published U.S. patent publication US 2013-0096152, which is hereby incorporated by reference in its entirety

Compound A may be synthesized as disclosed in U.S. Pat. No. 8,324,391.

Compound A may be formulated in a variety of dosage forms for administration by several routes of administration.

Leukotriene Modifiers

Cysteinyl leukotrienes mediate tissue edema, infiltration and activation of inflammatory cells. They are potent inflammatory mediators, produced by the 5-lipoxygenase pathway of arachidonic acid metabolism, and are believed to play a role in the pathophysiology of asthma by mediating bronchoconstriction and inflammatory reactions throughout different cells. Leukotriene modifiers include cysteinyl-leukotriene 1 receptor antagonists (montelukast, pranlukast, and zifurkulast) and a 5-lipoxygenase inhibitor (zileuton).

1. Montelukast

The chemical name of montelukast is

[R-(E)]-1-[[[1-[3-[2-(7-chloro-2-quinolinyl)ethenyl]phenyl]-3-[2-(1-hydroxy-1-methylethyl)phenyl]propyl]thio]methyl]cyclopropaneacetic acid. Its empirical formula is C₃₅H₃₆ClNO₃S and the molecular weight is 586.2.

Montelukast is used in medicaments mainly in the form of its monosodium salt. Montelukast, respectively, montelukast sodium, is a selective and orally active leukotriene receptor antagonist that inhibits the cysteinyl leukotriene CysLT1 receptor.

The structural formula of montelukast sodium is:

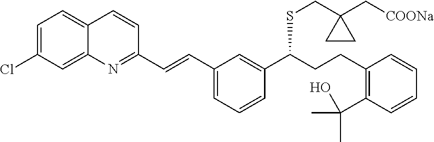

Montelukast and a method for its synthesis are disclosed in U.S. Pat. No. 5,565,473, which is hereby incorporated by reference in its entirety.

As pharmaceutically acceptable salts of montelukast may be mentioned montelukast monosodium salt, montelukast 1,2-ethandisulfonic acid salt and montelukast N,N′-dibenzylethylenediamine salt. A particularly preferred pharmaceutically acceptable salt of montelukast is the monosodium salt of montelukast.

Montelukast sodium and its preparation in amorphous as well as in crystalline form are disclosed in U.S. Pat. Nos. 5,565,473 and 5,614,632. Crystalline forms of montelukast sodium are disclosed in U.S. Pat. No. 6,320,052 (WO95/18107) and U.S. Pat. No. 7,560,559 (WO2004/091618). Crystalline montelukast free acid as well as an improved process for the preparation of amorphous montelukast sodium are disclosed in WO2004/108679 (U.S. patent application No. 2007-0082925). Crystalline 1,2-ethanedisulfonic acid and N,N′-dibenzylethylenediamine salts of montelukast are disclosed in WO2009/052625 (U.S. patent application No. 2010-0305080). Each of these patents and published U.S. patent applications, respectively, is hereby incorporated by reference, in its entirety.

Montelukast may be formulated in a variety of dosage forms for administration by several routes of administration. Montelukast sodium is formulated in granule form, tablet form and chewable tablet form, and in dosages of 4 mg, 5 mg and 10 mg. The preparation of Montelukast sodium oral granules is disclosed in U.S. Pat. No. 8,007,830 (WO2003/035036), hereby incorporated by reference in its entirety. A tablet composition containing 51.9 mg montelukast sodium is described in WO97/16173.

Label and Dosage Information: Montelukast sodium is marketed, inter alia, for prophylaxis and chronic treatment of asthma in patients 12 months of age and older. Montelukast sodium should be taken in the evening. The recommended doses are one 10 mg tablet for adults and adolescents 15 years of age and older; one 5 mg chewable tablet for pediatric patients 6 to 14 years of age; one 4 mg chewable tablet or one packet of 4 mg oral granules for pediatric patients 2 to 5 years of age; and one packet of 4 mg oral granules for pediatric patients 12 to 23 months of age.

2. Pranlukast

The chemical name of pranlukast is N-[4-oxo-2-(1H-tetrazol-5-yl)-4H-chromen-7-yl]-4-(4-phenylbutoxy)benzamide. Its empirical formula is C₂₇H₂₃N₅O₄ and the molecular weight is 481.503. Like montelukast, pranlukast is an orally active leukotriene receptor antagonist that inhibits the cysteinyl leukotriene CysLT1 receptor.

The structural formula of pranlukast is:

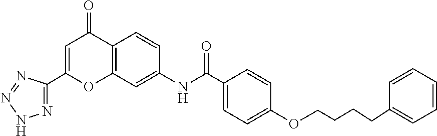 [SMILES: O=C(Nc1ccc2c(=O)cc(-c3nn[nH]n3)oc2c1)c1ccc(OCCCCc2ccccc2)cc1]

Pranlukast and a method for its synthesis are disclosed in U.S. Pat. No. 4,780,463, which is hereby incorporated by reference in its entirety.

As pharmaceutically acceptable salts of pranlukast may be mentioned pranlukast sodium salt.

In WO2006/109737 pranlukast hemi-hydrate containing pharmaceutical preparations (tablets quickly disintegrating in the oral cavity) are disclosed, exhibiting a reduced bitterness. In WO2007/060802 further pharmaceutical formulations are disclosed containing a stable pranlukast hydrate.

Label and Dosage information: Pranlukast hydrate is marketed in Japan and other countries for prophylaxis and chronic treatment of bronchial asthma and allergic rhinitis in adults and children. In Japan, Pranlukast hydrate should be taken in the morning and the evening. The recommended doses are 225 mg capsules twice daily (two 112.5 mg-capsules per once, twice daily) for adults 15 years of age and older and 35 mg/kg of 10% dry syrup twice daily for children 14 years of age and younger.

3. Zafirlukast

The chemical name of zafirlukast is

Cyclopentyl 3-{2-methoxy-4-[(o-tolylsulfonyl)carbamoyl]benzyl}-1-methyl-1H-indol-5-ylcarbamate. Its empirical formula is O₃₁H₃₃N₃O₆S and the molecular weight is 575.676. Like montelukast, zafirlukast is an orally active leukotriene receptor antagonist that inhibits the cysteinyl leukotriene CysLT1 receptor.

The structural formula of zafirlukast is:

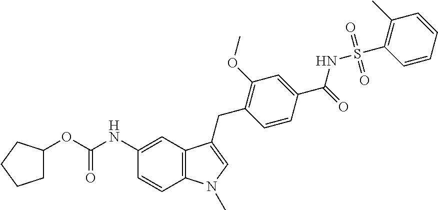 [SMILES: COc1cc(C(=O)NS(=O)(=O)c2ccccc2C)ccc1Cc1cn(C)c2ccc(NC(=O)OC3CCCC3)cc12]

Zafirlukast and a method for its synthesis are disclosed in U.S. Pat. No. 4,859,692, which is hereby incorporated by reference in its entirety.

In EP0490648 and EP0490649 (U.S. Pat. Nos. 5,319,097, 5,482,963 and 5,993,859) new crystalline polymorphic forms of zafirlukast and pharmaceutical formulations including these new crystalline polymorphic forms are disclosed. The patent disclosures referenced above are hereby incorporated by reference in their entirety.

Label and Dosage Information: Zafirlukast is currently marketed for the prophylaxis and chronic treatment of asthma in adults and children 5 years of age and older. The current recommended oral dose of Zafirlukast in the United States in adults and children 12 years of age and older is 20 mg tablets twice daily. The current recommended dose of Zafirlukast in children 5 through 11 years of age is 10 mg tablets twice daily.

4. Zileuton

The chemical name of zileuton is N-[1-(1-benzothien-2-yl)ethyl]-N-hydroxyurea. Its empirical formula is C₁₁H₁₂N₂O₂S and the molecular weight is 236.291. Zileuton is an orally active inhibitor of 5-lipoxygenase, and thus inhibits leukotrienes (LTB₄, LTC₄, LTD₄, and LTE₄) formation.

The structural formula of zileuton is:

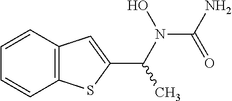 [SMILES: CC(c1cc2ccccc2s1)N(O)C(N)=O]

Zileuton and a method for its synthesis are disclosed in U.S. Pat. No. 4,873,259, which is hereby incorporated by reference in its entirety.

Label and Dosage Information: Zileuton (controlled release) is marketed for the prophylaxis and chronic treatment of asthma in adults and children 12 years of age or older. The recommended oral dose of Zileuton (controlled release) is two 600 mg extended release tablets twice daily, for a total daily dose of 2400 mg.

Detailed Info about Standard Medication for Severe Asthma According to GINA 2011 Report and GINA 2009 Children Report

In one aspect (aspect a) the present invention relates to a method of treating adult and adolescent (15 years of age and older) patients suffering from partly controlled or uncontrolled severe asthma, not adequately controlled despite treatment with a rapid-acting β2-agonist on an as needed basis and maintenance treatment with medium- or high dose inhaled glucocorticosteroids plus long-acting β2-agonists.

Suitable rapid-acting β2-agonists which may be mentioned in connection with aspect a) by way of example, include salbutamol, terbutaline, fenoterol, levalbuterol HFA, reproterol and pirbuterol.

Suitable long-acting inhaled β2-agonists which may be mentioned in connection with aspect a) by way of example, include formoterol and salmeterol.

Suitable long-acting oral β2-agonists which may be mentioned in connection with aspect a) by way of example, include slow release formulations of salbutamol, terbutaline and bambuterol.

Suitable inhaled glucocorticosteriods (ICS) which may be mentioned in connection with aspect a) by way of example, include beclomethasone dipropionate (CFC; >500-1000/>1000-2000), beclomethasone dipropionate (HFA; >250-500/>500-1000), budesonide (≧400-800/>800-1600), ciclesonide (>160-320/>320-1280), flunisolide (>1000-2000/>2000), fluticasone propionate (>250-500/>500-1000), mometasone furoate (400/800) and triamcinolone acetonide (>1000-2000/>2000). The first number or range in the parentheses behind the indicated pharmaceutical name is the medium daily dose in mcg for the respective ICS for adult and adolescent (15 years of age and older) patients—the second number or range indicates the high daily dose in mcg for such ICS for adult and adolescent (15 years of age and older) patients—according to GINA 2011 Report.

In another aspect (aspect b) the present invention relates to a method of treating pediatric patients older than 5 years of age and younger than 15 years of age suffering from partly controlled or uncontrolled severe asthma, not adequately controlled despite treatment with a rapid-acting β2-agonist on an as needed basis and maintenance treatment with medium- or high-dose inhaled glucocorticosteroids plus long-acting β2-agonists.

Suitable rapid-acting β2-agonists which may be mentioned in connection with aspect b) by way of example include primarily inhaled rapid-acting β2-agonists, such as include salbutamol, fenoterol, levalbuterol HFA and reproterol.

Suitable long-acting inhaled β2-agonists which may be mentioned in connection with aspect b) by way of example, include formoterol and salmeterol.

Suitable inhaled glucocorticosteriods (ICS) which may be mentioned in connection with aspect b) by way of example, include beclomethasone dipropionate (>200-400/>400), budesonide (>200-400/>400), ciclesonide (>160-320/>320), flunisolide (>750-1250/>1250), fluticasone propionate (>200-500/>500), mometasone furoate (≧200/≧400) and triamcinolone acetonide (>800-1200/>1200). The first number or range in the parentheses behind the indicated pharmaceutical name is the medium daily dose in mcg for the respective ICS for pediatric patients older than 5 years of age and younger than 15 years of age—the second number or range indicates the high daily dose in mcg for such ICS for pediatric patients older than 5 years of age and younger than 15 years of age—according to GINA 2011 Report.

In a further aspect (aspect c) the present invention relates to a method of treating pediatric patients 5 years of age and younger suffering from partly controlled or uncontrolled severe asthma, not adequately controlled despite treatment with rapid-acting β2-agonist on an as needed basis and maintenance treatment with double low dose inhaled glucocorticosteroid.

Suitable rapid-acting β2-agonists which may be mentioned in connection with aspect c) by way of example include primarily inhaled rapid-acting β2-agonists, such as salbutamol, fenoterol, levalbuterol HFA and reproterol.

Suitable inhaled glucocorticosteriods (ICS) which may be mentioned in connection with aspect c) by way of example, include beclomethasone dipropionate (100), budesonide MDI spacer (200), budesonide nebulized (500) and fluticasone propionate (100). The number in the parentheses behind the indicated pharmaceutical name is the low daily dose in mcg for the respective ICS for pediatric patients 5 years of age and younger—according to GINA 2009 Children Report.

In a further aspect (aspect d) the present invention relates to a method of treating adult patients suffering from partly controlled or uncontrolled severe asthma, not adequately controlled despite treatment with a rapid-acting β2-agonist on an as needed basis and maintenance treatment with high-dose inhaled glucocorticosteroids plus long-acting β2-agonists plus 5 mcg tiotropium bromide mono hydrate.

In connection with aspect d) the 5 mcg tiotropium bromide mono hydrate are preferable administered to the patient suffering from severe asthma by taking 2 puffs from the Spiriva® Respimat®.

Add-on Mono Therapy

Compound A or a pharmaceutically acceptable salt thereof may be used as add-on treatment in case adequate control of severe asthma cannot be achieved by administration of the recommended standard medication according to GINA 2011 Report or GINA 2009 Children Report.

Compound A or a pharmaceutically acceptable salt thereof may be administered by a variety of routes. Administration can be, for example, pulmonary, oral, parenteral or transdermal. The preferred route of administration is oral.

The preferred dosage form for mono therapy is an oral dosage form. Suitable oral dosage forms include tablets, capsules, powders, pills, solutions, suspensions, emulsions, pastes and granules. The most preferred oral dosage form is a tablet.

Dosage Information for Add-on Mono Therapy

The PDE4 inhibitor (in particular Compound A) may be administered once daily, twice daily, three times a day or four times a day. Once daily administration is particularly preferred and may take place preferably in the morning or in the evening.

Compound A may be present in an oral dosage form intended for once daily administration in an amount from 0.05 to 3.0 mg, such as, but not limited to 0.05, 0.075, 0.1, 0.125, 0.15, 0.175, 0.2, 0.25, 0.3, 0.35, 0.4, 0.45, 0.5, 0.75, 1.0, 1.25, 1.5, 1.75, 2.0, 2.5 or 3.0 mg; in one embodiment of the invention Compound A may be present in an oral dosage form intended for once daily administration in an amount from 1 to 3 mg, such as, but not limited to 1.0, 1.25, 1.5, 1.75, 2.0, 2.5, or 3.0 mg. In further embodiments of the invention Compound A may be present in an oral dosage form intended for once daily administration depending on the age of the patient to be treated in an amount from 0.15 to 1.0 mg, 0.1 to 0.5 mg or 0.05 to 0.2 mg. Specific dosages, which may be mentioned in this connection by way of example are 0.05, 0.075, 0.1, 0.125, 0.15, 0.175, 0.2, 0.25, 0.3, 0.35, 0.4, 0.45, 0.5, 0.75 or 1.0 mg.

Compound A may be present in an oral dosage form intended for once daily administration for adult patients and adolescents patients 15 years of age and older in an amount from 0.15 to 3.0 mg, such as, but not limited to 0.150, 0.175, 0.2, 0.25, 0.3, 0.35, 0.4, 0.45, 0.5, 0.75, 1.0, 1.25, 1.5, 1.75, 2.0, 2.5 or 3.0 mg. In a further embodiment of the invention Compound A may be present in an oral dosage form intended for once daily administration for adult patients and adolescents patients 15 years of age and older in an amount from 1 to 3 mg, such as, for example, 1.0, 1.25, 1.5, 1.75, 2.0, 2.5 or 3.0 mg. In yet a further embodiment of the invention Compound A may be present in an oral dosage form intended for once daily administration for adult patients and adolescents patients 15 years of age and older in an amount from 0.15 to 1 mg, such as, but not limited to 0.15, 0.175, 0.2, 0.25, 0.3, 0.35, 0.4, 0.45, 0.5, 0.75 or 1 mg.

In an oral dosage form intended for once daily administration for pediatric patients older than 5 and younger than 15 years of age Compound A may be present in an amount from 0.1 to 2 mg, such as, but not limited to 0.1, 0.125, 0.150, 0.175, 0.2, 0.25, 0.3, 0.35, 0.4, 0.45, 0.5, 0.75, 1.0, 1.25, 1.5, 1.75 or 2.0 mg. In an embodiment of the invention Compound A may be present in an oral dosage form intended for once daily administration for pediatric patients older than 5 and younger than 15 years of age in an amount from 0.5 to 2 mg, such as, but not limited to 0.5, 0.75, 1.0, 1.25, 1.5, 1.75 or 2.0. In another embodiment of the invention Compound A may be present in an oral dosage form intended for once daily administration for pediatric patients older than 5 and younger than 15 years of age in an amount from 0.1 to 0.5 mg, such as, but not limited to 0.1, 0.125, 0.150, 0.175, 0.2, 0.25, 0.3, 0.35, 0.4, 0.45 or 0.5 mg.

Compound A may be present in the oral dosage form intended for once daily administration for pediatric patients 5 years of age and younger in an amount from 0.05 to 1.5 mg, such as, but not limited to 0.05, 0.075, 0.1, 0.125, 0.150, 0.175, 0.2, 0.25, 0.3, 0.35, 0.4, 0.45, 0.5, 0.75, 1.0, 1.25 or 1.5 mg. In an embodiment of the invention Compound A may be present in the oral dosage form intended for once daily administration for pediatric patients 5 years of age and younger in an amount from 0.5 to 1.5 mg, such as, but not limited to 0.5, 0.75, 1.0, 1.25 or 1.5 mg. In another embodiment of the invention Compound A may be present in the oral dosage form intended for once daily administration for pediatric patients 5 years of age and younger in an amount from 0.05 to 0.2 mg, such as, but not limited to 0.05, 0.075, 0.1, 0.125, 0.150, 0.175 or 0.2 mg.

If a twice daily administration is intended instead of a once daily administration the above indicated amounts of Compound A can be divided in half.

Corresponding amounts of a pharmaceutically acceptable salt of Compound A can easily be calculated by one of ordinary skill, depending on the choice of the respective salt.

Add-on Combination Therapy

As an alternative to mono therapy, a combination of Compound A or a pharmaceutically acceptable salt thereof and a leukotriene modifier or a pharmaceutically acceptable salt thereof may be used as add-on treatment in case adequate control of severe asthma cannot be achieved by administration of the recommended standard medication according to GINA 2011 Report or GINA 2009 Children Report.

For combination therapy Compound A or a pharmaceutically acceptable salt thereof, may be co-administered with the leukotriene modifier or pharmaceutically acceptable salts thereof, concurrently, concomitantly or sequentially. Compound A or a pharmaceutically acceptable salt thereof, may be co-administered with the leukotriene modifier or a pharmaceutically acceptable salt thereof, by the same or different route(s) of administration. Compound A or a pharmaceutically acceptable salt thereof, may be co-administered with the leukotriene modifier or a pharmaceutically acceptable salt thereof, in the same or different formulations, including, but not limited to:

a) a single oral dosage form containing both Compound A or a pharmaceutically acceptable salt thereof, and the leukotriene modifier or a pharmaceutically acceptable salt thereof;

b) two separate oral dosage forms wherein one oral dosage form contains Compound A or a pharmaceutically acceptable salt thereof and the other oral dosage form contains the leukotriene modifier or a pharmaceutically acceptable salt thereof;

c) a single transdermal dosage form containing both Compound A or a pharmaceutically acceptable salt thereof and the leukotriene modifier or a pharmaceutically acceptable salt thereof;

d) two separate transdermal dosage forms wherein one transdermal dosage form contains Compound A or a pharmaceutically acceptable salt thereof and the other transdermal dosage form contains the leukotriene modifier or a pharmaceutically acceptable salt thereof;

e) a single intravenous dosage form containing both Compound A or a pharmaceutically acceptable salt thereof and the leukotriene modifier or a pharmaceutically acceptable salt thereof;

f) two separate intravenous dosage forms wherein one intravenous dosage form contains Compound A or a pharmaceutically acceptable salt thereof and the other intravenous dosage form contains the leukotriene modifier or a pharmaceutically acceptable salt thereof;

g) a single inhalative dosage form containing both Compound A or a pharmaceutically acceptable salt thereof and the leukotriene modifier or a pharmaceutically acceptable salt thereof;

h) two separate inhalative dosage forms wherein one inhalative dosage form contains Compound A or a pharmaceutically acceptable salt thereof and the other inhalative dosage form contains the leukotriene modifier or a pharmaceutically acceptable salt thereof; and

i) two separate dosage forms wherein the first dosage form contains Compound A or a pharmaceutically acceptable salt thereof and the second dosage form contains the leukotriene modifier or a pharmaceutically acceptable salt thereof and wherein the first and the second dosage form are administered by different routes of administration.

The preferred dosage form for combination therapy is a single oral dosage form providing administration of both Compound A or a pharmaceutically acceptable salt thereof and the leukotriene modifier or a pharmaceutically acceptable salt thereof. Suitable oral dosage forms include tablets, capsules, powders, pills, solutions, suspensions, emulsions, pastes and granules. The most preferred oral dosage forms for combination therapy include tablets, each tablet containing both Compound A or a pharmaceutically acceptable salt thereof and the leukotriene modifier or a pharmaceutically acceptable salt thereof.

Dosage information for combination treatment

The combination of the PDE4 inhibitor and the leukotriene modifier may be co-administered, depending on the particular combination, once daily, twice daily, three times a day or four times a day.

In case of a combination of Compound A and montelukast once daily co-administration is particularly preferred. Once daily co-administration may take place preferably in the morning or in the evening.

The oral dosage forms for once daily co-administration of a combination of Compound A and montelukast, may be either in form of
a) a single oral dosage form, which contains both Compound A and montelukast; or in form of
b) two separated oral dosage forms, in which one dosage form contains Compound A and the other dosage form contains montelukast.

Montelukast may be present in any amount from 0.1 to 50 mg, such as 0.1, 0.25, 0.5, 1, 1.5, 2, 3, 4, 5, 6, 7, 8, 9, 10, 15, 20, 25, 30 and 50 mg; more preferably, in an amount of 1, 2, 4, 5, 8, 10 or 20 mg. Compound A may be present in an amount from 0.05 to 3.0 mg, such as, but not limited to 0.05, 0.075, 0.1, 0.125, 0.150, 0.175, 0.2, 0.25, 0.3, 0.35, 0.4, 0.45, 0.5, 0.75, 1.0, 1.25, 1.5, 1.75, 2.0, 2.5 or 3.0 mg; in an embodiment of the invention Compound A may be present in an amount of 1 to 3 mg, such as, but not limited to 1.0, 1.25, 1.5, 1.75, 2.0, 2.5 or 3.0 mg; in further embodiments of the invention Compound A may be present depending on the age of the patient to be treated in an amount from 0.15 to 1 mg, 0.1 to 0.5 mg or 0.05 to 0.2 mg. Specific dosages, which may be mentioned in this connection by way of example are 0.05, 0.075, 0.1, 0.125, 0.15, 0.175, 0.2, 0.25, 0.3, 0.35, 0.4, 0.45, 0.5, 0.75 or 1.0 mg.

The amount of Compound A and montelukast, respectively, in the oral dosage form intended for once daily co-administration for adult patients and adolescents patients 15 years of age and older may be selected from 3.0 mg/15 mg; 2.5 mg/15 mg; 2.0 mg/15 mg; 1.75 mg/15 mg; 1.5 mg/15 mg; 1.25 mg/15 mg; 1.0 mg/15 mg; 0.75 mg/15 mg; 0.5 mg/15 mg; 0.45 mg/15 mg; 0.4 mg/15 mg; 0.35 mg/15 mg; 0.3 mg/15 mg; 0.25 mg/15 mg; 0.2 mg/15 mg; 0.175 mg/15 mg; 0.150 mg/15 mg; 3.0 mg/10 mg; 2.5 mg/10 mg; 2.0 mg/10 mg; 1.75 mg/10 mg; 1.5 mg/10 mg; 1.25 mg/10 mg; 1.0 mg/10 mg; 0.75 mg/10 mg; 0.5 mg/10 mg; 0.45 mg/10 mg; 0.4 mg/10 mg; 0.35 mg/10 mg; 0.3 mg/10 mg; 0.25 mg/10 mg; 0.2 mg/10 mg; 0.175 mg/10 mg; 0.15 mg/10 mg; 3.0 mg/8 mg; 2.5 mg/8 mg; 2.0 mg/8 mg; 1.75 mg/8 mg; 1.5 mg/8 mg; 1.25 mg/8 mg; 1.0 mg/8 mg; 0.75 mg/8 mg; 0.5 mg/8 mg; 0.45 mg/8 mg; 0.4 mg/8 mg; 0.35 mg/8 mg; 0.3 mg/8 mg; 0.25 mg/8 mg; 0.2 mg/8 mg; 0.175 mg/8 mg or 0.15 mg/8 mg; (amount of Compound A/amount of Montelukast).

The amount of Compound A and montelukast, respectively, in the oral dosage form intended for once daily co-administration for pediatric patients older than 5 and younger than 15 years of age may be selected from 2.0 mg/10 mg; 1.75 mg/10 mg; 1.5 mg/10 mg; 1.25 mg/10 mg; 1.0 mg/10 mg; 0.75 mg/10 mg; 0.5 mg/10 mg; 0.45 mg/10 mg; 0.4 mg/10 mg; 0.35 mg/10 mg; 0.3 mg/10 mg; 0.25 mg/10 mg; 0.2 mg/10 mg; 0.175 mg/10 mg; 0.15 mg/10 mg; 0.125 mg/10 mg; 0.1 mg/10 mg; 2.0 mg/8 mg; 1.75 mg/8 mg; 1.5 mg/8 mg; 1.25 mg/8 mg; 1.0 mg/8 mg; 0.75 mg/8 mg; 0.5 mg/8 mg; 0.45 mg/8 mg; 0.4 mg/80 mg; 0.35 mg/8 mg; 0.3 mg/8 mg; 0.25 mg/8 mg; 0.2 mg/8 mg; 0.175 mg/8 mg; 0.15 mg/8 mg; 0.125 mg/8 mg; 0.1 mg/8 mg; 2.0 mg/5 mg; 1.75 mg/5 mg; 1.5 mg/5 mg; 1.25 mg/5 mg; 1.0 mg/5 mg; 0.75 mg/5 mg; 0.5 mg/5 mg; 0.45 mg/5 mg; 0.4 mg/5 mg; 0.35 mg/5 mg; 0.3 mg/5 mg; 0.25 mg/5 mg; 0.2 mg/5 mg; 0.175 mg/5 mg; 0.15 mg/5 mg; 0.125 mg/5 mg or 0.1 mg/5 mg (amount of Compound A/amount of Montelukast).

The amount of Compound A and montelukast, respectively, in the oral dosage form intended for once daily co-administration for pediatric patients 5 years of age and younger may be selected from 1.5 mg/5 mg; 1.25 mg/5 mg; 1.0 mg/5 mg; 0.75 mg/5 mg; 0.5 mg/5 mg; 0.45 mg/5 mg; 0.4 mg/5 mg; 0.35 mg/5 mg; 0.3 mg/5 mg; 0.25 mg/5 mg; 0.2 mg/5 mg; 0.175 mg/5 mg; 0.15 mcg/5 mg; 0.125 mg/5 mg; 0.1 mg/5 mg; 0.075 mg/5 mg; 0.05 mg/5 mg; 1.5 mg/4 mg; 1.25 mg/4 mg; 1.0 mg/4 mg; 0.75 mg/4 mg; 0.5 mg/4 mg; 0.45 mg/4 mg; 0.4 mg/4 mg; 0.35 mg/4 mg; 0.3 mg/4 mg; 0.25 mg/4 mg 0.2 mg/4 mg; 0.175 mg/4 mg; 0.15 mcg/4 mg; 0.125 mg/4 mg; 0.1 mg/4 mg; 0.075 mg/4 mg; 0.05 mg/4 mg; 1.5 mg/3 mg; 1.25 mg/3 mg; 1.0 mg/3 mg; 0.75 mg/3 mg; 0.5 mg/3 mg; 0.45 mg/3 mg; 0.4 mg/3 mg; 0.35 mg/3 mg; 0.3 mg/3 mg; 0.25 mg/3 mg; 0.2 mg/3 mg; 0.175 mg/3 mg; 0.15 mcg/3 mg; 0.125 mg/3 mg; 0.1 mg/3 mg; 0.075 mg/3 mg; or 0.05 mg/3 mg (amount of Compound A/amount of Montelukast).

If a twice daily co-administration is intended instead of a once daily administration the above indicated amounts of Compound A and montelukast can be divided in half.

Corresponding amounts of a pharmaceutically acceptable salt of Compound A and/or montelukast can easily be calculated by one of ordinary skill, depending on the choice of the respective salt.

Pharmaceutical Formulations and Dosage Forms

When employed as pharmaceuticals, the compounds of the invention can be administered in the form of pharmaceutical compositions. These compositions can be prepared in a manner well known in the pharmaceutical art, and can be administered by a variety of routes. Administration can be pulmonary (e.g., by inhalation or insufflation of powders or aerosols, including by nebulizer; intratracheal, intranasal, epidermal and transdermal), oral or parenteral. Parenteral administration includes intravenous, subcutaneous, intraperitoneal intramuscular or injection or infusion. Parenteral administration can be in the form of a single bolus dose, or can be, for example, by a continuous perfusion pump. Pharmaceutical compositions and formulations for topical administration can include transdermal patches. Conventional pharmaceutical carriers, aqueous, powder or oily bases, thickeners and the like may be necessary or desirable.

This invention also includes pharmaceutical compositions which contain, as the active ingredient, one or more of the compounds above in combination with one or more pharmaceutically acceptable carriers. In making the compositions of the invention, the active ingredients are typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), soft and hard gelatin capsules, suppositories, sterile injectable solutions, and sterile packaged powders.

In preparing a formulation, each active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing an amount of each active ingredient as described above. The term “unit dosage form” refers to a physically discrete unit suitable as a unitary dosage for human subjects and other mammals, each unit containing a predetermined quantity of active material calculated to produce the desired therapeutic effect, in association with a suitable pharmaceutical excipient. An oral dosage form is a preferred unit dosage form.

The active compounds can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient(s) is (are) mixed with a pharmaceutical excipient to form a solid preformulation composition containing a homogeneous mixture of the active ingredient(s). When referring to these preformulation compositions as homogeneous, the active ingredient(s) is (are) typically dispersed evenly throughout the composition so that the composition can be readily subdivided into equally effective unit dosage forms such as tablets, pills and capsules. This solid preformulation is then subdivided into unit dosage forms of the type described above.

The tablets or pills of the present invention can be coated or otherwise compounded to provide a dosage form affording the advantage of prolonged action. For example, the tablet or pill can include an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described above. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

Pre-Clinical Studies

The effect of either a) mono-administration of Compound A or alternatively b) co-administration of Compound A and montelukast in comparison to administration of either Compound A or montelukast alone may be tested, for example, in a steroid-refractory, neutrophilic antigen-induced severe asthma model in mice.

One suitable mouse model has recently been described by Essilfie, Foster, Hansbro et al in Thorax 2012 (Thorax 67: 588-99, 2012; Title: Combined Haemophilus influenzae respiratory infection and allergic airways disease drives chronic infection and features of neutrophilic asthma). Neutrophilic asthma represents one of the phenotypes found in partly controlled or uncontrolled severe asthma. Given that the gram-negative pathogen (non-typeable) Haemophilus influenza (NTHi) is found in the airways of 60% of patients afflicted with neutrophilic asthma, a mouse model in which allergen sensitization and challenge are preceded by airway NTHi infection has been developed. Briefly, in this model, female Balb/c mice are inoculated intratracheally with NTHi (500.000 CFU). 10 days after infection mice are intraperitoneally sensitized with ovalbumin (OVA, 50 mcg) along with aluminium hydroxide adjuvants. Mice are then exposed to repeated, once daily intranasally administered challenges with ovalbumin (10 mcg per 50 μl saline) at days 12, 13, 14 and 15.

In this experimental model, when assessed the day after the last ovalbumin challenge there is a significant increase in neutrophils accumulating in the bronchoalveolar lavage (BAL) in the NTHi/OVA-treated mice compared to the non-infected OVA-treated animals. As shown by Essilfie, Foster, Hansbro et al in this model, while dexamethasone (1 mg kg-1, intranasal) almost abolishes the BAL neutrophils in the non-infected, OVA-treated mice, the steroid does not affect the number of BAL neutrophils in the NTHi-infected, OVA-treated mice, reflecting a key differentiator between mild-to-moderate, steroid-responsive asthma and severe, neutrophilic asthma that is refractory to glucocorticosteroids.

Parallel findings were reported for IL-17 released from cultured mediastinal lymph node T-cells. In addition, NTHi infection imparts loss of steroid-responsiveness for BAL eosinophils and IL-5 as well as IL-13 release from T-cells of mediastinal lymph nodes as well as airway hyperreactivity to methacholine (based on body plethysmographic measurements of dynamic compliance and transpulmonary resistance).

When Compound A is administered alone or Compound A and montelukast are co-administered over the period of allergen challenge an expected outcome would be a suppression of neutrophilic airway inflammation as well as airway hyperreactivity to methacholine opposite to what has been observed with dexamethasone.

In the above described model of neutrophilic, allergen-induced asthma, NTHi inoculation results in a chronic infection.

A further suitable model to test the effects of co-administration of Compound A and montelukast on steroid-refractory, neutrophilic antigen-induced severe asthma may be generated by a modification of the above-described model. The mice of the above-described model may—instead of being infected with NTHi—be co-exposed to LPS, a constituent of gram-negative bacteria walls over the period of allergen challenge. As a result, as with the model described above, a less steroid responsive neutrophilic airway inflammation and steroid resistant airway hyperreactivity to methacholine may evolve, as indicated by Yang et al. in J Immunol 182: 5107-15, 2009.

Still a further suitable model to test the effects of a) mono-administration of Compound A or alternatively b) co-administration of Compound A and montelukast in comparison to administration of either Compound A or montelukast alone is the steroid insensitive severe asthma model in mice recently described by Charles River/Argenta at the ERS congress in Munich 2014. Briefly, in this model female BALB/c mice are sensitized subcutaneously on day 0 with 100₄ of house dust mite (HDM) in Complete Freud's Adjuvant (CFA). On day 14 mice are exposed to saline or 100 μg HDM by the intranasal (i.n.) route (in a modification of this model mice may exposed to 100 μg HDM on days 14 and 15). Test compound administration can be started prior to or after HDM challenge. Twenty four hours following HDM challenge inflammatory cells are counted in the bronchoalveolar fluid (in a modification of this model inflammatory cell count in the bronchoalveolar fluid may be done 48 h following HDM challenge. The model exhibits both neutrophilic and eosinophilic inflammation, histopathological changes in the lungs and elevated levels of Th-1/Th-2 cytokines, all of which are relatively insensitive to inhaled steroids. This model may be representative of the mixed TH1 (ILC1)/TH2 (ILC2)/TH17 (ILC2) orchestrated airway inflammation found in patients afflicted with severe asthma. Neutralizing IFNγ mitigates AHR but augments eosinophilic and neutrophilic airway inflammation along with IL-17 and HDM specific IgE. In contrast neither neutralizing IL-4 nor IL-17 affects AHR. Blocking IL-4 mitigates eosinophilic airway inflammation and HDM specific IgE while blocking IL-17 alleviates neutrophilic inflammation but does not affect eosinophils or HDM specific IgE. If this translates into man, therapies broadly hitting this mixed immunology would be required to effectively manage severe asthma (Dixon et al; Journal of Inflammation 10 (Suppl 1) P7, 2013).

When Compound A is administered alone or the Compound A and montelukast are co-administered over the period of allergen challenge an expected outcome in this model would be a suppression of neutrophilic airway inflammation opposite to what has been observed with steroids.

As suitable readouts for the effect of either a) the mono-administration of the PDE4 inhibitor (in particular Compound A) or alternatively b) the co-administration of the PDE4 inhibitor (in particular Compound A) and the leukotriene modifier (in particular montelukast) in the above described animal models may be mentioned, for example, (a) bronchoalveolar lavage (BAL) inflammatory cell counts and (b) lung functions, such as for example, compliance, FVC and airway hyperresponsiveness (AHR), (c) mucus hypersecretion (MUC5AC) and (d) lung interstitial inflammation.

Further Aspects of the Invention

While it is expected that the administration of either a) a PDE4 inhibitor alone or b) a combination of a PDE4 inhibitor and a leukotriene modifier has the most pronounced effects in patients with partly controlled or uncontrolled severe asthma, the administration of either a) a PDE4 inhibitor alone or b) a combination of a PDE4 inhibitor and a leukotriene modifier may also have beneficial effects in patients with partly controlled or uncontrolled milder forms of asthma. Accordingly, with reference to the GINA Step levels discussed above, the administration of either a) a PDE4 inhibitor alone or b) a PDE4 inhibitor and a leukotriene modifier may also be useful in patients suffering from partly controlled or uncontrolled mild to moderate asthma, not adequately controlled despite treatment with
a) a rapid-acting β2-agonist on a as needed basis and maintenance treatment with a low dose inhaled glucocorticosteroid; or
b) a rapid-acting β2-agonist on a as needed basis and maintenance treatment with a medium dose inhaled glucocorticosteroid; or
c) a rapid-acting β2-agonist on a as needed basis and maintenance treatment with a high dose inhaled glucocorticosteroid; or
d) a rapid-acting β2-agonist on a as needed basis and maintenance treatment with a low dose inhaled glucocorticosteroid plus a long-acting β2-agonist.

In addition, the administration of either a) a PDE4 inhibitor alone or b) a combination of a PDE4 inhibitor and a leukotriene modifier may also have beneficial effects in patients with partly controlled or uncontrolled very severe forms of asthma. Accordingly, the administration of either a) a PDE4 inhibitor alone or b) a PDE4 inhibitor and a leukotriene modifier may also be useful in patients suffering from partly controlled or uncontrolled asthma, not adequately controlled despite treatment with
a) a rapid-acting β2-agonist on a as needed basis and maintenance treatment with a medium or high dose inhaled glucocorticosteroid plus a long-acting β2-agonist and a low dose oral glucocorticosteroid; or
b) a rapid-acting β2-agonist on a as needed basis and maintenance treatment with a medium or high dose inhaled glucocorticosteroid plus a long-acting β2-agonist and anti-IgE treatment.

The invention also relates to a method of
improving lung function (FEV₁, FVC, FEF25-75 or PEF);
reducing the number of daytime symptoms;
reducing the number of nocturnal symptoms;
reducing the use of short-acting β2-agonists;
decreasing the frequency, lengthen the time to or shorten the duration of a moderate exacerbation;
decreasing the frequency, lengthen the time to or shorten the duration of a severe exacerbation; or
reducing the dose of the inhaled glucocorticosteroid and/or of the long-acting β2-agonist in a patient suffering from partly controlled or uncontrolled severe asthma, not adequately controlled despite treatment with a rapid-acting β2-agonist on a as needed basis and maintenance treatment with a medium or high dose inhaled glucocorticosteroid (ICS) plus a long-acting β2-agonist, including administering to the patient a therapeutically effective amount of either a) a phosphodiesterase 4 (PDE4) inhibitor or b) a phosphodiesterase 4 (PDE4) inhibitor and a leukotriene modifier.

Other further aspects of the invention are:
a) Pharmaceutical composition comprising a phosphodiesterase 4 (PDE4) inhibitor for use in the treatment of partly controlled severe asthma in a patient whose asthma is not adequately controlled despite treatment according to GINA Step 4.
b) Pharmaceutical composition comprising a phosphodiesterase 4 (PDE4) inhibitor for use in the treatment of uncontrolled severe asthma in a patient whose asthma is not adequately controlled despite treatment according to GINA Step 4.
c) Pharmaceutical composition comprising a phosphodiesterase 4 (PDE4) inhibitor for use in the treatment of partly controlled severe asthma in a patient whose asthma is not adequately controlled despite treatment with a medium dose inhaled glucocorticosteroid (ICS) plus a long-acting β2 agonist.
d) Pharmaceutical composition comprising a phosphodiesterase 4 (PDE4) inhibitor for use in the treatment of uncontrolled severe asthma in a patient whose asthma is not adequately controlled despite treatment with a medium dose inhaled glucocorticosteroid (ICS) plus a long-acting β2 agonist.
e) Pharmaceutical composition comprising a phosphodiesterase 4 (PDE4) inhibitor for use in the treatment of partly controlled severe asthma in a patient whose asthma is not adequately controlled despite treatment with a rapid-acting β2 agonist on a as needed basis and maintenance treatment with a medium dose inhaled glucocorticosteroid (ICS) plus a long-acting β2 agonist.
f) Pharmaceutical composition comprising a phosphodiesterase 4 (PDE4) inhibitor for use in the treatment of uncontrolled severe asthma in a patient whose asthma is not adequately controlled despite treatment with a rapid-acting β2 agonist on a as needed basis and maintenance treatment with a medium dose inhaled glucocorticosteroid (ICS) plus a long-acting β2 agonist.
g) Pharmaceutical composition comprising a phosphodiesterase 4 (PDE4) inhibitor for use in the treatment of partly controlled severe asthma in a patient whose asthma is not adequately controlled despite treatment with a high dose inhaled glucocorticosteroid (ICS) plus a long-acting β2 agonist.
h) Pharmaceutical composition comprising a phosphodiesterase 4 (PDE4) inhibitor for use in the treatment of uncontrolled severe asthma in a patient whose asthma is not adequately controlled despite treatment with a high dose inhaled glucocorticosteroid (ICS) plus a long-acting β2 agonist.
i) Pharmaceutical composition comprising a phosphodiesterase 4 (PDE4) inhibitor for use in the treatment of partly controlled severe asthma in a patient whose asthma is not adequately controlled despite treatment with a rapid-acting β2 agonist on a as needed basis and maintenance treatment with a high dose inhaled glucocorticosteroid (ICS) plus a long-acting 82 agonist.
j) Pharmaceutical composition comprising a phosphodiesterase 4 (PDE4) inhibitor for use in the treatment of uncontrolled severe asthma in a patient whose asthma is not adequately controlled despite treatment with a rapid-acting β2 agonist on a as needed basis and maintenance treatment with a high dose inhaled glucocorticosteroid (ICS) plus a long-acting 82 agonist.
k) Pharmaceutical composition according to any one of a) to j), wherein the patient is selected from one or more of: adult patient, adolescent patient 15 years of age and older and pediatric patient older than 5 years and younger than 15 years of age.
l) Pharmaceutical composition according to any one of a) to j), wherein the patient is an adult patient.
m) Pharmaceutical composition according to any one of a), c), e), g) and i), wherein partly controlled means that the patient to be treated suffers from at least one symptom selected from the group consisting of
   a) daytime cough, wheeze or dyspnea, experienced more than twice a week;
   b) any limitation on daily activities;
   c) nocturnal cough, wheeze or dyspnea or asthma-related awakening;
   d) need for reliever/rescue treatment more than twice a week and
   e) lung function (PEF or FEV₁) of less than 80% of the predicted value (without administration of a bronchodilator).
n) Pharmaceutical composition according to any one of b), d), f), h) and j), wherein uncontrolled means that the patient to be treated suffers from three or more symptoms selected from the group consisting of
   a) daytime cough, wheeze or dyspnea, experienced more than twice a week;
   b) any limitation on daily activities;
   c) nocturnal cough, wheeze or dyspnea or asthma-related awakening;
   d) need for reliever/rescue treatment more than twice a week and
   e) lung function (PEF or FEV₁) of less than 80% of the predicted value (without administration of a bronchodilator).
o) Pharmaceutical composition comprising a phosphodiesterase 4 (PDE4) inhibitor for use in the treatment of partly controlled severe asthma in a pediatric patient 5 years of age or younger whose asthma is not adequately controlled despite treatment with double low dose inhaled glucocorticosteroid (ICS).
p) Pharmaceutical composition comprising a phosphodiesterase 4 (PDE4) inhibitor for use in the treatment of uncontrolled severe asthma in a pediatric patient 5 years of age or younger whose asthma is not adequately controlled despite treatment with double low dose inhaled glucocorticosteroid (ICS).
q) Pharmaceutical composition comprising a PDE4 inhibitor for use in the treatment of partly controlled severe asthma in a pediatric patient 5 years of age or younger whose asthma is not adequately controlled despite treatment with a rapid-acting β2-agonist on an as-needed basis and maintenance treatment with a double low dose inhaled glucocorticosteroid (ICS).
r) Pharmaceutical composition comprising a phosphodiesterase 4 (PDE4) inhibitor for use in the treatment of uncontrolled severe asthma in a pediatric patient 5 years of age or younger whose asthma is not adequately controlled despite treatment with a rapid-acting β2-agonist on an as-needed basis and maintenance treatment with a double low dose inhaled glucocorticosteroid (ICS).
s) Pharmaceutical composition according to any one of o) and q), wherein partly controlled means that the pediatric patient to be treated suffers from at least one symptom selected from the group consisting of
   a) daytime wheezing, cough or difficult breathing experienced more than twice a week;
   b) any limitation of daily activities;
   c) nocturnal cough, wheezing, difficult breathing or asthma related awakening and
   d) need for reliever/rescue treatment more than 2 days a week.
t) Pharmaceutical composition according to any one of p) and r), wherein uncontrolled means that the pediatric patient to be treated suffers from more than three symptoms selected from the group consisting of
   a) daytime wheezing, cough or difficult breathing experienced more than twice a week;
   b) any limitation of daily activities;
   c) nocturnal cough, wheezing, difficult breathing or asthma related awakening and
   d) need for reliever/rescue treatment more than 2 days a week.
u) Pharmaceutical composition comprising a phosphodiesterase 4 (PDE4) inhibitor for use in the treatment of partly controlled or uncontrolled severe asthma.
v) Pharmaceutical composition according to any one of a) to j), l) to n), and u), wherein the phosphodiesterase 4 (PDE4) inhibitor is selected from the group consisting of Compound A and a pharmaceutically acceptable salt of Compound A.
w) Pharmaceutical composition according to any one of a) to j), l to n), and u), wherein the phosphodiesterase 4 (PDE4) inhibitor is Compound A.
x) Pharmaceutical composition according to any one of o) to t), wherein the phosphodiesterase 4 (PDE4) inhibitor is selected from the group consisting of Compound A and a pharmaceutically acceptable salt of Compound A.
y) Pharmaceutical composition according to any one of o) to t), wherein the phosphodiesterase 4 (PDE4) inhibitor is Compound A.
z) Pharmaceutical composition according to any one of v) and w), wherein the phosphodiesterase 4 (PDE4) inhibitor is in an oral dosage form.
aa) Pharmaceutical composition according to any one of x) and y), wherein the phosphodiesterase 4 (PDE4) inhibitor is in an oral dosage form.
bb) Pharmaceutical dosage form according to z), wherein the oral dosage form includes an amount of Compound A selected from 1.0, 1.25, 1.5, 1.75, 2.0, 2.5 and 3.0 mg or an amount of a pharmaceutically acceptable salt of Compound A corresponding to 1.0, 1.25, 1.5, 1.75, 2.0, 2.5 or 3.0 mg of Compound A.
cc) Pharmaceutical dosage form according to z), wherein the oral dosage form includes an amount of Compound A selected from 0.150, 0.175, 0.2, 0.25, 0.3, 0.35, 0.4, 0.45, 0.5, 0.75 and 1.0 mg or an amount of a pharmaceutically acceptable salt of Compound A corresponding to 0.150, 0.175, 0.2, 0.25, 0.3, 0.35, 0.4, 0.45, 0.5, 0.75 or 1.0 mg of Compound A.
dd) Pharmaceutical composition comprising a phosphodiesterase 4 (PDE4) inhibitor and a leukotriene modifier for use in the treatment of partly controlled severe asthma in a patient whose asthma is not adequately controlled despite treatment according to GINA Step 4.
ee) Pharmaceutical composition comprising a phosphodiesterase 4 (PDE4) inhibitor and a leukotriene modifier for use in the treatment of uncontrolled severe asthma in a patient whose asthma is not adequately controlled despite treatment according to GINA Step 4.
ff) Pharmaceutical composition comprising a phosphodiesterase 4 (PDE4) inhibitor and a leukotriene modifier for use in the treatment of partly controlled severe asthma in a patient whose asthma is not adequately controlled despite treatment with a medium dose inhaled glucocorticosteroid (ICS) plus a long-acting β2 agonist.
gg) Pharmaceutical composition comprising a phosphodiesterase 4 (PDE4) inhibitor and a leukotriene modifier for use in the treatment of uncontrolled severe asthma in a patient whose asthma is not adequately controlled despite treatment with a medium dose inhaled glucocorticosteroid (ICS) plus a long-acting β2 agonist.
hh) Pharmaceutical composition comprising a phosphodiesterase 4 (PDE4) inhibitor and a leukotriene modifier for use in the treatment of partly controlled severe asthma in a patient whose asthma is not adequately controlled despite treatment with a rapid-acting β2 agonist on a as needed basis and maintenance treatment with a medium dose inhaled glucocorticosteroid (ICS) plus a long-acting β2 agonist.
ii) Pharmaceutical composition comprising a phosphodiesterase 4 (PDE4) inhibitor and a leukotriene modifier for use in the treatment of uncontrolled severe asthma in a patient whose asthma is not adequately controlled despite treatment with a rapid-acting β2 agonist on a as needed basis and maintenance treatment with a medium dose inhaled glucocorticosteroid (ICS) plus a long-acting β2 agonist.
jj) Pharmaceutical composition comprising a phosphodiesterase 4 (PDE4) inhibitor and a leukotriene modifier for use in the treatment of partly controlled severe asthma in a patient whose asthma is not adequately controlled despite treatment with a high dose inhaled glucocorticosteroid (ICS) plus a long-acting β2 agonist.
kk) Pharmaceutical composition comprising a phosphodiesterase 4 (PDE4) inhibitor and a leukotriene modifier for use in the treatment of uncontrolled severe asthma in a patient whose asthma is not adequately controlled despite treatment with a high dose inhaled glucocorticosteroid (ICS) plus a long-acting β2 agonist.
ll) Pharmaceutical composition comprising a phosphodiesterase 4 (PDE4) inhibitor and a leukotriene modifier for use in the treatment of partly controlled severe asthma in a patient whose asthma is not adequately controlled despite treatment with a rapid-acting β2 agonist on a as needed basis and maintenance treatment with a high dose inhaled glucocorticosteroid (ICS) plus a long-acting β2 agonist.
mm) Pharmaceutical composition comprising a phosphodiesterase 4 (PDE4) inhibitor and a leukotriene modifier for use in the treatment of uncontrolled severe asthma in a patient whose asthma is not adequately controlled despite treatment with a rapid-acting β2 agonist on a as needed basis and maintenance treatment with a high dose inhaled glucocorticosteroid (ICS) plus a long-acting β2 agonist.
nn) Pharmaceutical composition according to any one of dd) to mm), wherein the patient is selected from one or more of: adult patient, adolescent patient 15 years of age and older and pediatric patient older than 5 years and younger than 15 years of age.
oo) Pharmaceutical composition according to any one of dd) to mm), wherein the patient is an adult patient.
pp) Pharmaceutical composition according to any one of dd), ff), hh), jj) and ll), wherein partly controlled means that the patient to be treated suffers from at least one symptom selected from the group consisting of
   a) daytime cough, wheeze or dyspnea, experienced more than twice a week;
   b) any limitation on daily activities;
   c) nocturnal cough, wheeze or dyspnea or asthma-related awakening;
   d) need for reliever/rescue treatment more than twice a week and
   e) lung function (PEF or FEV₁) of less than 80% of the predicted value (without administration of a bronchodilator).
qq) Pharmaceutical composition according to any one of ee), gg), ii), kk) and mm), wherein uncontrolled means that the patient to be treated suffers from three or more symptoms selected from the group consisting of
   a) daytime cough, wheeze or dyspnea, experienced more than twice a week;
   b) any limitation on daily activities;
   c) nocturnal cough, wheeze or dyspnea or asthma-related awakening;
   d) need for reliever/rescue treatment more than twice a week and
   e) lung function (PEF or FEV₁) of less than 80% of the predicted value (without administration of a bronchodilator).
rr) Pharmaceutical composition comprising a phosphodiesterase 4 (PDE4) inhibitor and a leukotriene modifier for use in the treatment of partly controlled severe asthma in a pediatric patient 5 years of age or younger whose asthma is not adequately controlled despite treatment with double low dose inhaled glucocorticosteroid (ICS).
ss) Pharmaceutical composition comprising a phosphodiesterase 4 (PDE4) inhibitor and a leukotriene modifier for use in the treatment of uncontrolled severe asthma in a pediatric patient 5 years of age or younger whose asthma is not adequately controlled despite treatment with double low dose inhaled glucocorticosteroid (ICS).
tt) Pharmaceutical composition comprising a PDE4 inhibitor and a leukotriene modifier for use in the treatment of partly controlled severe asthma in a pediatric patient 5 years of age or younger whose asthma is not adequately controlled despite treatment with a rapid-acting β2-agonist on an as-needed basis and maintenance treatment with a double low dose inhaled glucocorticosteroid (ICS).
uu) Pharmaceutical composition comprising a phosphodiesterase 4 (PDE4) inhibitor and a leukotriene modifier for use in the treatment of uncontrolled severe asthma in a pediatric patient 5 years of age or younger whose asthma is not adequately controlled despite treatment with a rapid-acting β2-agonist on an as-needed basis and maintenance treatment with a double low dose inhaled glucocorticosteroid (ICS).
vv) Pharmaceutical composition according to any one of rr) and tt), wherein partly controlled means that the pediatric patient to be treated suffers from at least one symptom selected from the group consisting of
   a) daytime wheezing, cough or difficult breathing experienced more than twice a week;
   b) any limitation of daily activities;
   c) nocturnal cough, wheezing, difficult breathing or asthma related awakening and
   d) need for reliever/rescue treatment more than 2 days a week.
ww) Pharmaceutical composition according to any one of ss) and uu), wherein uncontrolled means that the pediatric patient to be treated suffers from more than three symptoms selected from the group consisting of
   a) daytime wheezing, cough or difficult breathing experienced more than twice a week;
   b) any limitation of daily activities;
   c) nocturnal cough, wheezing, difficult breathing or asthma related awakening and
   d) need for reliever/rescue treatment more than 2 days a week.
xx) Pharmaceutical composition comprising a phosphodiesterase 4 (PDE4) inhibitor and a leukotriene modifier for use in the treatment of partly controlled or uncontrolled severe asthma.
yy) Pharmaceutical composition according to any one of dd) to mm), oo) to qq), and ww), wherein the phosphodiesterase 4 (PDE4) inhibitor and the leukotriene modifier are in one single dosage form.
zz) Pharmaceutical composition according to any one of dd) to mm), oo) to qq), and ww), wherein the phosphodiesterase 4 (PDE4) inhibitor and the leukotriene modifier are in one single oral dosage form.
aaa) Pharmaceutical composition according to any one of rr) to ww), wherein the phosphodiesterase 4 (PDE4) inhibitor and the leukotriene modifier are in one single dosage form.
bbb) Pharmaceutical composition according to any one of rr) to ww), wherein the phosphodiesterase 4 (PDE4) inhibitor and the leukotriene modifier are in one single oral dosage form.
ccc) Pharmaceutical composition according to any one of dd) to mm), oo) to qq), and ww), wherein the phosphodiesterase 4 (PDE4) inhibitor and the leukotriene modifier are in two separate dosage forms.
ddd) Pharmaceutical composition according to any one of dd) to mm), oo) to qq), and ww), wherein the phosphodiesterase 4 (PDE4) inhibitor and the leukotriene modifier are in two separate oral dosage forms.
eee) Pharmaceutical composition according to any one of rr) to ww), wherein the phosphodiesterase 4 (PDE4) inhibitor and the leukotriene modifier are in two separate dosage forms.
fff) Pharmaceutical composition according to any one of rr) to ww), wherein the phosphodiesterase 4 (PDE4) inhibitor and the leukotriene modifier are in two separate oral dosage forms.
ggg) Pharmaceutical composition according to ccc), wherein the phosphodiesterase 4 (PDE4) inhibitor and the leukotriene modifier are in two separate dosage forms and the two separate dosage forms are administered concurrently.
hhh) Pharmaceutical composition according to ddd), wherein the phosphodiesterase 4 (PDE4) inhibitor and the leukotriene modifier are in two separate oral dosage forms and the two separate oral dosage forms are administered concurrently.
iii) Pharmaceutical composition according to eee), wherein the phosphodiesterase 4 (PDE4) inhibitor and the leukotriene modifier are in two separate dosage forms and the two separate dosage forms are administered concurrently.
jjj) Pharmaceutical composition according to fff), wherein the phosphodiesterase 4 (PDE4) inhibitor and the leukotriene modifier are in two separate oral dosage forms and the two separate oral dosage forms are administered concurrently.
kkk) Pharmaceutical composition according to any one of yy), zz), ccc), ddd), ggg) and hhh), wherein the phosphodiesterase 4 (PDE4) inhibitor is selected from the group consisting of Compound A and a pharmaceutically acceptable salt of Compound A.
lll) Pharmaceutical composition according to any one of yy), zz), ccc), ddd), ggg) and hhh), wherein the phosphodiesterase 4 (PDE4) inhibitor is Compound A.
mmm) Pharmaceutical composition according to any one of aaa), bbb), eee), fff), iii) and jjj), wherein the phosphodiesterase 4 (PDE4) inhibitor is selected from the group consisting of Compound A and a pharmaceutically acceptable salt of Compound A.
nnn) Pharmaceutical composition according to any one of aaa), bbb), eee), fff), iii) and jjj), wherein the phosphodiesterase 4 (PDE4) inhibitor is Compound A.
ooo) Pharmaceutical composition according to any one of kkk) and lll), wherein the leukotriene modifier is selected from the group consisting of montelukast, pranlukast, zafirlukast, zileuton and a pharmaceutically acceptable salt thereof.
ppp) Pharmaceutical composition according to any one of kkk) and lll), wherein the leukotriene modifier is montelukast or a pharmaceutically acceptable salt thereof.
qqq) Pharmaceutical composition according to any one of kkk) and lll), wherein the leukotriene modifier is montelukast sodium.
rrr) Pharmaceutical composition according to any one of mmm) and nnn), wherein the leukotriene modifier is selected from the group consisting of montelukast, pranlukast, zafirlukast, zileuton and a pharmaceutically acceptable salt thereof.
sss) Pharmaceutical composition according to any one of mmm) and nnn), wherein the leukotriene modifier is montelukast or a pharmaceutically acceptable salt thereof.
ttt) Pharmaceutical composition according to any one of mmm) and nnn), wherein the leukotriene modifier is montelukast sodium.
uuu) Pharmaceutical composition according to hhh), wherein
   a) the oral dosage form comprising the phosphodiesterase 4 (PDE4) inhibitor contains Compound A in an amount selected from 1.0, 1.25, 1.5, 1.75, 2.0, 2.5 and 3.0 mg;
   b) the oral dosage form comprising the leukotriene modifier contains montelukast sodium in an amount corresponding to 10 mg montelukast and
   c) the pharmaceutical composition is administered once daily.
vvv) Pharmaceutical composition according to hhh), wherein
   a) the oral dosage form comprising the phosphodiesterase 4 (PDE4) inhibitor contains Compound A in an amount selected from 0.150, 0.175, 0.2, 0.25, 0.3, 0.35, 0.4, 0.45, 0.5, 0.75 and 1.0 mg;
   b) the oral dosage form comprising the leukotriene modifier contains montelukast sodium in an amount corresponding to 10 mg montelukast and
   c) the pharmaceutical composition is administered once daily.
www) Pharmaceutical composition according to zz), wherein the one single oral dosage form includes an amount of Compound A selected from 1.0, 1.25, 1.5, 1.75, 2.0, 2.5 and 3.0 mg and an amount of montelukast sodium corresponding to 10 mg of montelukast.
xxx) Pharmaceutical composition according to zz), wherein the one single oral dosage form includes an amount of Compound A selected from 0.150, 0.175, 0.2, 0.25, 0.3, 0.35, 0.4, 0.45, 0.5, 0.75 and 1.0 mg and an amount of montelukast sodium corresponding to 10 mg of montelukast.
yyy) Pharmaceutical composition comprising a phosphodiesterase 4 (PDE4) inhibitor and a leukotriene modifier.
zzz) Pharmaceutical composition according to yyy), wherein the phosphodiesterase 4 (PDE4) inhibitor and the leukotriene modifier are in one single dosage form.
aaaa) Pharmaceutical composition according to yyy), wherein the phosphodiesterase 4 (PDE4) inhibitor and the leukotriene modifier are in one single oral dosage form.
bbbb) Pharmaceutical composition according to yyy), wherein the phosphodiesterase 4 (PDE4) inhibitor and the leukotriene modifier are in two separate dosage forms.
cccc) Pharmaceutical composition according to yyy) wherein the phosphodiesterase 4 (PDE4) inhibitor and the leukotriene modifier are in two separate oral dosage forms.
dddd) Pharmaceutical composition according to bbbb), wherein the phosphodiesterase 4 (PDE4) inhibitor and the leukotriene modifier are in two separate dosage forms and the two separate dosage forms are administered concurrently.
eeee) Pharmaceutical composition according to cccc), wherein the phosphodiesterase 4 (PDE4) inhibitor and the leukotriene modifier are in two separate oral dosage forms and the two separate oral dosage forms are administered concurrently.
ffff) Pharmaceutical composition according to any one of yyy) to eeee), wherein the phosphodiesterase 4 (PDE4) inhibitor is selected from the group consisting of Compound A and a pharmaceutically acceptable salt of Compound A.
gggg) Pharmaceutical composition according to any one of yyy) to eeee), wherein the phosphodiesterase 4 (PDE4) inhibitor is Compound A.
hhhh) Pharmaceutical composition according to any one of yyy) to gggg), wherein the leukotriene modifier is selected from the group consisting of montelukast, pranlukast, zafirlukast, zileuton and a pharmaceutically acceptable salt thereof.
iiii) Pharmaceutical composition according to any one of yyy) to gggg), wherein the leukotriene modifier is montelukast or a pharmaceutically acceptable salt thereof.
jjjj) Pharmaceutical composition according to any one of yyy) to gggg), wherein the leukotriene modifier is montelukast sodium.
kkkk) Pharmaceutical composition according to cccc), wherein
   a) the oral dosage form comprising the phosphodiesterase 4 (PDE4) inhibitor contains Compound A in an amount selected from 1.0, 1.25, 1.5, 1.75, 2.0, 2.5 and 3.0 mg;
   b) the oral dosage form comprising the leukotriene modifier contains montelukast sodium in an amount corresponding to 10 mg montelukast and
   c) the pharmaceutical composition is administered once daily.
llll) Pharmaceutical composition according to cccc), wherein
   a) the oral dosage form comprising the phosphodiesterase 4 (PDE4) inhibitor contains Compound A in an amount selected from 0.150, 0.175, 0.2, 0.25, 0.3, 0.35, 0.4, 0.45, 0.5, 0.75 and 1.0 mg;
   b) the oral dosage form comprising the leukotriene modifier contains montelukast sodium in an amount corresponding to 10 mg montelukast and
   c) the pharmaceutical composition is administered once daily.
mmmm) Use of a phosphodiesterase 4 (PDE4) inhibitor for the manufacture of a pharmaceutical composition for the treatment of partly controlled severe asthma in a patient whose asthma is not adequately controlled despite treatment according to GINA Step 4.
nnnn) Use of a phosphodiesterase 4 (PDE4) inhibitor for the manufacture of a pharmaceutical composition for the treatment of uncontrolled severe asthma in a patient whose asthma is not adequately controlled despite treatment according to GINA Step 4.
oooo) Use of a phosphodiesterase 4 (PDE4) inhibitor for the manufacture of a pharmaceutical composition for the treatment of partly controlled severe asthma in a patient whose asthma is not adequately controlled despite treatment with a medium dose inhaled glucocorticosteroid (ICS) plus a long-acting β2 agonist.
pppp) Use of a phosphodiesterase 4 (PDE4) inhibitor for the manufacture of a pharmaceutical composition for the treatment of uncontrolled severe asthma in a patient whose asthma is not adequately controlled despite treatment with a medium dose inhaled glucocorticosteroid (ICS) plus a long-acting β2 agonist.
qqqq) Use of a phosphodiesterase 4 (PDE4) inhibitor for the manufacture of a pharmaceutical composition for the treatment of partly controlled severe asthma in a patient whose asthma is not adequately controlled despite treatment with a rapid-acting β2 agonist on a as needed basis and maintenance treatment with a medium dose inhaled glucocorticosteroid (ICS) plus a long-acting β2 agonist.
rrrr) Use of a phosphodiesterase 4 (PDE4) inhibitor for the manufacture of a pharmaceutical composition for the treatment of uncontrolled severe asthma in a patient whose asthma is not adequately controlled despite treatment with a rapid-acting β2 agonist on a as needed basis and maintenance treatment with a medium dose inhaled glucocorticosteroid (ICS) plus a long-acting β2 agonist.
ssss) Use of a phosphodiesterase 4 (PDE4) inhibitor for the manufacture of a pharmaceutical composition for the treatment of partly controlled severe asthma in a patient whose asthma is not adequately controlled despite treatment with a high dose inhaled glucocorticosteroid (ICS) plus a long-acting β2 agonist.
tttt) Use of a phosphodiesterase 4 (PDE4) inhibitor for the manufacture of a pharmaceutical composition for the treatment of uncontrolled severe asthma in a patient whose asthma is not adequately controlled despite treatment with a high dose inhaled glucocorticosteroid (ICS) plus a long-acting β2 agonist.
uuuu) Use of a phosphodiesterase 4 (PDE4) inhibitor for the manufacture of a pharmaceutical composition for the treatment of partly controlled severe asthma in a patient whose asthma is not adequately controlled despite treatment with a rapid-acting β2 agonist on a as needed basis and maintenance treatment with a high dose inhaled glucocorticosteroid (ICS) plus a long-acting β2 agonist.
vvvv) Use of a phosphodiesterase 4 (PDE4) inhibitor for the manufacture of a pharmaceutical composition for the treatment of uncontrolled severe asthma in a patient whose asthma is not adequately controlled despite treatment with a rapid-acting β2 agonist on a as needed basis and maintenance treatment with a high dose inhaled glucocorticosteroid (ICS) plus a long-acting β2 agonist.
wwww) Use according to any one of mmmm) to vvvv), wherein the patient is selected from one or more of: adult patient, adolescent patient 15 years of age and older and pediatric patient older than 5 years and younger than 15 years of age.
xxxx) Use according to any one of mmmm) to vvvv), wherein the patient is an adult patient.
yyyy) Use according to any one of mmmm), oooo), qqqq), ssss) and uuuu), wherein partly controlled means that the patient to be treated suffers from at least one symptom selected from the group consisting of
   a) daytime cough, wheeze or dyspnea, experienced more than twice a week;
   b) any limitation on daily activities;
   c) nocturnal cough, wheeze or dyspnea or asthma-related awakening;
   d) need for reliever/rescue treatment more than twice a week and
   e) lung function (PEF or FEV₁) of less than 80% of the predicted value (without administration of a bronchodilator).
zzzz) Use according to any one of nnnn), pppp), ), rrrr) and vvvv), wherein uncontrolled means that the patient to be treated suffers from three or more symptom selected from the group consisting of
   a) daytime cough, wheeze or dyspnea, experienced more than twice a week;
   b) any limitation on daily activities;
   c) nocturnal cough, wheeze or dyspnea or asthma-related awakening;
   d) need for reliever/rescue treatment more than twice a week and
   e) lung function (PEF or FEV₁) of less than 80% of the predicted value (without administration of a bronchodilator).
aaaaa) Use of a phosphodiesterase 4 (PDE4) inhibitor for the manufacture of a pharmaceutical composition for the treatment of partly controlled severe asthma in a pediatric patient 5 years of age or younger whose asthma is not adequately controlled despite treatment with double low dose inhaled glucocorticosteroid (ICS).
bbbbb) Use of a phosphodiesterase 4 (PDE4) inhibitor for the manufacture of a pharmaceutical composition for the treatment of uncontrolled severe asthma in a pediatric patient 5 years of age or younger whose asthma is not adequately controlled despite treatment with double low dose inhaled glucocorticosteroid (ICS).
ccccc) Use of a phosphodiesterase 4 (PDE4) inhibitor for the manufacture of a pharmaceutical composition for the treatment of partly controlled severe asthma in a pediatric patient 5 years of age or younger whose asthma is not adequately controlled despite treatment with a rapid-acting β2-agonist on an as-needed basis and maintenance treatment with a double low dose inhaled glucocorticosteroid (ICS).
ddddd) Use of a phosphodiesterase 4 (PDE4) inhibitor for the manufacture of a pharmaceutical composition for the treatment of uncontrolled severe asthma in a pediatric patient 5 years of age or younger whose asthma is not adequately controlled despite treatment with a rapid-acting β2-agonist on an as-needed basis and maintenance treatment with a double low dose inhaled glucocorticosteroid (ICS).
eeeee) Use according to any one of aaaaa) and ccccc), wherein partly controlled means that the pediatric patient to be treated suffers from at least one symptom selected from the group consisting of
   a) daytime wheezing, cough or difficult breathing experienced more than twice a week;
   b) any limitation of daily activities;
   c) nocturnal cough, wheezing, difficult breathing or asthma related awakening and
   d) need for reliever/rescue treatment more than 2 days a week.
fffff) Use according to any one of nnnn) and pppp), wherein uncontrolled means that the pediatric patient to be treated suffers from more than three symptoms selected from the group consisting of
   a) daytime wheezing, cough or difficult breathing experienced more than twice a week;
   b) any limitation of daily activities;
   c) nocturnal cough, wheezing, difficult breathing or asthma related awakening and
   d) need for reliever/rescue treatment more than 2 days a week.
ggggg) Use according to any one of mmmm) to vvvv) and xxxx) to zzzz), wherein the phosphodiesterase 4 (PDE4) inhibitor is selected from the group consisting of Compound A and a pharmaceutically acceptable salt of Compound A.
hhhhh) Use according to any one of mmmm) to vvvv) and xxxx) to zzzz), wherein the phosphodiesterase 4 (PDE4) inhibitor is Compound A.
iiiii) Use according to any one of aaaaa) to fffff), wherein the phosphodiesterase 4 (PDE4) inhibitor is selected from the group consisting of Compound A and a pharmaceutically acceptable salt of Compound A.
jjjjj) Use according to any one of aaaaa) to fffff), wherein the phosphodiesterase 4 (PDE4) inhibitor is Compound A.
kkkkk) Use according to any one of ggggg) and hhhhh), wherein the phosphodiesterase 4 (PDE4) inhibitor is in an oral dosage form.
lllll) Use according to any one of iiiii) and jjjjj), wherein the phosphodiesterase 4 (PDE4) inhibitor is in an oral dosage form.
mmmmm) Use according to kkkkk), wherein the oral dosage form includes an amount of Compound A selected from 1.0, 1.25, 1.5, 1.75, 2.0, 2.5 and 3.0 mg or an amount of a pharmaceutically acceptable salt of Compound A corresponding to 1.0, 1.25, 1.5, 1.75, 2.0, 2.5 or 3.0 mg of Compound A.
nnnnn) Use according to kkkkk), wherein the oral dosage form includes an amount of Compound A selected from 0.150, 0.175, 0.2, 0.25, 0.3, 0.35, 0.4, 0.45, 0.5, 0.75 and 1.0 mg or an amount of a pharmaceutically acceptable salt of Compound A corresponding to 0.150, 0.175, 0.2, 0.25, 0.3, 0.35, 0.4, 0.45, 0.5, 0.75 or 1.0 mg of Compound A.
ooooo) Use of a phosphodiesterase 4 (PDE4) inhibitor and a leukotriene modifier for the manufacture of a pharmaceutical composition for the treatment of partly controlled severe asthma in a patient whose asthma is not adequately controlled despite treatment according to GINA Step 4.
ppppp) Use of a phosphodiesterase 4 (PDE4) inhibitor and a leukotriene modifier for the manufacture of a pharmaceutical composition for the treatment of uncontrolled severe asthma in a patient whose asthma is not adequately controlled despite treatment according to GINA Step 4.
qqqqq) Use of a phosphodiesterase 4 (PDE4) inhibitor and a leukotriene modifier for the manufacture of a pharmaceutical composition for the treatment of partly controlled severe asthma in a patient whose asthma is not adequately controlled despite treatment with a medium dose inhaled glucocorticosteroid (ICS) plus a long-acting β2 agonist.
rrrrr) Use of a phosphodiesterase 4 (PDE4) inhibitor and a leukotriene modifier for the manufacture of a pharmaceutical composition for the treatment of uncontrolled severe asthma in a patient whose asthma is not adequately controlled despite treatment with a medium dose inhaled glucocorticosteroid (ICS) plus a long-acting β2 agonist.
sssss) Use of a phosphodiesterase 4 (PDE4) inhibitor and a leukotriene modifier for the manufacture of a pharmaceutical composition for the treatment of partly controlled severe asthma in a patient whose asthma is not adequately controlled despite treatment with a rapid-acting β2 agonist on a as needed basis and maintenance treatment with a medium dose inhaled glucocorticosteroid (ICS) plus a long-acting β2 agonist.
ttttt) Use of a phosphodiesterase 4 (PDE4) inhibitor and a leukotriene modifier for the manufacture of a pharmaceutical composition for the treatment of uncontrolled severe asthma in a patient whose asthma is not adequately controlled despite treatment with a rapid-acting β2 agonist on a as needed basis and maintenance treatment with a medium dose inhaled glucocorticosteroid (ICS) plus a long-acting β2 agonist.
uuuuu) Use of a phosphodiesterase 4 (PDE4) inhibitor and a leukotriene modifier for the manufacture of a pharmaceutical composition for the treatment of partly controlled severe asthma in a patient whose asthma is not adequately controlled despite treatment with a high dose inhaled glucocorticosteroid (ICS) plus a long-acting β2 agonist.
vvvvv) Use of a phosphodiesterase 4 (PDE4) inhibitor and a leukotriene modifier for the manufacture of a pharmaceutical composition for the treatment of uncontrolled severe asthma in a patient whose asthma is not adequately controlled despite treatment with a high dose inhaled glucocorticosteroid (ICS) plus a long-acting β2 agonist.
wwwww) Use of a phosphodiesterase 4 (PDE4) inhibitor and a leukotriene modifier for the manufacture of a pharmaceutical composition for the treatment of partly controlled severe asthma in a patient whose asthma is not adequately controlled despite treatment with a rapid-acting β2 agonist on a as needed basis and maintenance treatment with a high dose inhaled glucocorticosteroid (ICS) plus a long-acting β2 agonist.
xxxxx) Use of a phosphodiesterase 4 (PDE4) inhibitor and a leukotriene modifier for the manufacture of a pharmaceutical composition for the treatment of uncontrolled severe asthma in a patient whose asthma is not adequately controlled despite treatment with a rapid-acting β2 agonist on a as needed basis and maintenance treatment with a high dose inhaled glucocorticosteroid (ICS) plus a long-acting β2 agonist.
yyyyy) Use according to any one of ooooo) to xxxxx), wherein the patient is selected from one or more of: adult patient, adolescent patient 15 years of age and older and pediatric patient older than 5 years and younger than 15 years of age.
zzzzz) Use according to any one of ooooo) to xxxxx), wherein the patient is an adult patient.
aaaaaa) Use according to any one of ooooo), qqqqq), sssss), uuuuu) and wwwww), wherein partly controlled means that the patient to be treated suffers from at least one symptom selected from the group consisting of
   a) daytime cough, wheeze or dyspnea, experienced more than twice a week;
   b) any limitation on daily activities;
   c) nocturnal cough, wheeze or dyspnea or asthma-related awakening;
   d) need for reliever/rescue treatment more than twice a week and
   e) lung function (PEF or FEV₁) of less than 80% of the predicted value (without administration of a bronchodilator).
bbbbbb) Use according to any one of ppppp), rrrrr), ttttt), vvvvv) and xxxxx), wherein uncontrolled means that the patient to be treated suffers from three or more symptom selected from the group consisting of
   a) daytime cough, wheeze or dyspnea, experienced more than twice a week;
   b) any limitation on daily activities;
   c) nocturnal cough, wheeze or dyspnea or asthma-related awakening;
   d) need for reliever/rescue treatment more than twice a week and
   e) lung function (PEF or FEV₁) of less than 80% of the predicted value (without administration of a bronchodilator).
cccccc) Use of a phosphodiesterase 4 (PDE4) inhibitor and a leukotriene modifier for the manufacture of a pharmaceutical composition for the treatment of partly controlled severe asthma in a pediatric patient 5 years of age or younger whose asthma is not adequately controlled despite treatment with double low dose inhaled glucocorticosteroid (ICS).
dddddd) Use of a phosphodiesterase 4 (PDE4) inhibitor and a leukotriene modifier for the manufacture of a pharmaceutical composition for the treatment of uncontrolled severe asthma in a pediatric patient 5 years of age or younger whose asthma is not adequately controlled despite treatment with double low dose inhaled glucocorticosteroid (ICS).
eeeeee) Use of a phosphodiesterase 4 (PDE4) inhibitor and a leukotriene modifier for the manufacture of a pharmaceutical composition for the treatment of partly controlled severe asthma in a pediatric patient 5 years of age or younger whose asthma is not adequately controlled despite treatment with a rapid-acting β2-agonist on an as-needed basis and maintenance treatment with a double low dose inhaled glucocorticosteroid (ICS).
ffffff) Use of a phosphodiesterase 4 (PDE4) inhibitor and a leukotriene modifier for the manufacture of a pharmaceutical composition for the treatment of uncontrolled severe asthma in a pediatric patient 5 years of age or younger whose asthma is not adequately controlled despite treatment with a rapid-acting β2-agonist on an as-needed basis and maintenance treatment with a double low dose inhaled glucocorticosteroid (ICS).
gggggg) Use according to any one of cccccc) and eeeeee), wherein partly controlled means that the pediatric patient to be treated suffers from at least one symptom selected from the group consisting of
   a) daytime wheezing, cough or difficult breathing experienced more than twice a week;
   b) any limitation of daily activities;
   c) nocturnal cough, wheezing, difficult breathing or asthma related awakening and
   d) need for reliever/rescue treatment more than 2 days a week.
hhhhhh) Use according to any one of dddddd) and ffffff), wherein uncontrolled means that the pediatric patient to be treated suffers from more than three symptoms selected from the group consisting of
   a) daytime wheezing, cough or difficult breathing experienced more than twice a week;
   b) any limitation of daily activities;
   c) nocturnal cough, wheezing, difficult breathing or asthma related awakening and
   d) need for reliever/rescue treatment more than 2 days a week.
iiiiii) Use of a phosphodiesterase 4 (PDE4) inhibitor and a leukotriene modifier for the manufacture of a pharmaceutical composition for the treatment of partly controlled or uncontrolled asthma.
jjjjjj) Use according to any one of ooooo) to xxxxx), zzzzz) to bbbbbb), and iiiiii), wherein the pharmaceutical composition comprises the phosphodiesterase 4 (PDE4) inhibitor and the leukotriene modifier in one single dosage form.
kkkkkk) Use according to any one of ooooo) to xxxxx), zzzzz) to bbbbbb) and iiiiii), wherein the pharmaceutical composition comprises the phosphodiesterase 4 (PDE4) inhibitor and the leukotriene modifier in one single oral dosage form.
llllll) Use according to any one of cccccc) to hhhhhh), wherein the pharmaceutical composition comprises the phosphodiesterase 4 (PDE4) inhibitor and the leukotriene modifier in one single dosage form.
mmmmmm) Use according to any one cccccc) to hhhhhh), wherein the pharmaceutical composition comprises the phosphodiesterase 4 (PDE4) inhibitor and the leukotriene modifier in one single oral dosage form.
nnnnnn) Use according to any one of oooooo) to xxxxx), zzzzz) to bbbbbb), and iiiiii), wherein the pharmaceutical composition comprises the phosphodiesterase 4 (PDE4) inhibitor and the leukotriene modifier in two separate dosage forms.
oooooo) Use according to any one of oooooo) to xxxxx), zzzzz) to bbbbbb), and iiiiii), wherein the pharmaceutical composition comprises the phosphodiesterase 4 (PDE4) inhibitor and the leukotriene modifier in two separate oral dosage forms.
pppppp) Use according to any one of cccccc) to hhhhhh), wherein the pharmaceutical composition comprises the phosphodiesterase 4 (PDE4) inhibitor and the leukotriene modifier in two separate dosage forms.
qqqqqq) Use according to any one of cccccc) to hhhhhh), wherein the pharmaceutical composition comprises the phosphodiesterase 4 (PDE4) inhibitor and the leukotriene modifier in two separate oral dosage forms.
rrrrrr) Use according to nnnnnn), wherein the two separate dosage forms are administered concurrently.
ssssss) Use according to pppppp), wherein the two separate dosage forms are administered concurrently.
tttttt) Use according to oooooo), wherein the two separate oral dosage forms are administered concurrently.
uuuuuu) Use according to qqqqqq), wherein the two separate oral dosage forms are administered concurrently.
vvvvvv) Use according to any one of jjjjjj), kkkkkk), nnnnnn), oooooo), rrrrrr) and tttttt), wherein the phosphodiesterase 4 (PDE4) inhibitor is selected from the group consisting of Compound A and a pharmaceutically acceptable salts of Compound A.
wwwwww) Use according to any one of jjjjjj), kkkkkk), nnnnnn), oooooo), rrrrrr) and tttttt), wherein the phosphodiesterase 4 (PDE4) inhibitor is Compound A.
xxxxxx) Use according to any one of llllll), mmmmmm), pppppp), qqqqqq), ssssss) and uuuuuu), wherein the phosphodiesterase 4 (PDE4) inhibitor is selected from the group consisting of Compound A and a, pharmaceutically acceptable salts of Compound A.
yyyyyy) Use according to any one of llllll), mmmmmm), pppppp), qqqqqq), ssssss) and uuuuuu), wherein the phosphodiesterase 4 (PDE4) inhibitor is Compound A.
zzzzzz) Use according to any one of vvvvvv) and wwwwww), wherein the leukotriene modifier is selected from the group consisting of montelukast, pranlukast, zafirlukast, zileuton and pharmaceutically acceptable salts thereof.
aaaaaaa) Use according to any one of vvvvvv) and wwwwww), wherein the leukotriene modifier is montelukast or a pharmaceutically acceptable salt thereof.
bbbbbbb) Use according to any one of vvvvvv) to wwwwww), wherein the leukotriene modifier is montelukast sodium.
ccccccc) Use according to any one of xxxxxx) and yyyyyy), wherein the leukotriene modifier is selected from the group consisting of montelukast, pranlukast, zafirlukast, zileuton and pharmaceutically acceptable salts thereof.
ddddddd) Use according to any one of xxxxxx) and yyyyyy), wherein the leukotriene modifier is montelukast or a pharmaceutically acceptable salt thereof.
eeeeeee) Use according to any one of xxxxxx) and yyyyyy), wherein the leukotriene modifier is montelukast sodium.
fffffff) Use according to tttttt), wherein
a) the oral dosage form comprising the phosphodiesterase 4 (PDE4) inhibitor contains Compound A in an amount selected from 1.0, 1.25, 1.5, 1.75, 2.0, 2.5 and 3.0 mg or an amount of a pharmaceutically acceptable salt of Compound A corresponding to 1.0, 1.25, 1.5, 1.75, 2.0, 2.5 or 3.0 mg of Compound A;
b) the oral dosage form comprising the leukotriene modifier contains montelukast sodium in an amount corresponding to 10 mg montelukast and
c) the pharmaceutical composition is administered once daily.
ggggggg) Use according to tttttt), wherein
a) the oral dosage form comprising the phosphodiesterase 4 (PDE4) inhibitor contains Compound A in an amount selected from 0.150, 0.175, 0.2, 0.25, 0.3, 0.35, 0.4, 0.45, 0.5, 0.75 and 1.0 mg or an amount of a pharmaceutically acceptable salt of Compound A corresponding to 0.150, 0.175, 0.2, 0.25, 0.3, 0.35, 0.4, 0.45, 0.5, 0.75 or 1.0 mg of Compound A;
b) the oral dosage form comprising the leukotriene modifier contains montelukast sodium in an amount corresponding to 10 mg montelukast and
c) the pharmaceutical composition is administered once daily.
hhhhhhh) Use according to kkkkkk), wherein the one single oral dosage form includes an amount of Compound A selected from 1.0, 1.25, 1.5, 1.75, 2.0, 2.5 and 3.0 mg or an amount of a pharmaceutically acceptable salt of Compound A corresponding to 1.0, 1.25, 1.5, 1.75, 2.0, 2.5 or 3.0 mg of Compound A and an amount of montelukast sodium corresponding to 10 mg of montelukast.
iiiiiii) Use according to kkkkkk), wherein the one single oral dosage form includes an amount of Compound A selected from 0.150, 0.175, 0.2, 0.25, 0.3, 0.35, 0.4, 0.45, 0.5, 0.75 and 1.0 mg or an amount of a pharmaceutically acceptable salt of Compound A corresponding to 0.150, 0.175, 0.2, 0.25, 0.3, 0.35, 0.4, 0.45, 0.5, 0.75 or 1.0 mg of Compound A and an amount of montelukast sodium corresponding to 10 mg of montelukast.
jjjjjjj) Use of a phosphodiesterase 4 (PDE4) inhibitor selected from the group consisting of Compound A and a pharmaceutically acceptable salt of Compound A for the manufacture of a pharmaceutical composition for the treatment of any one of
   (a) partly controlled severe asthma in an adult patient whose asthma is not adequately controlled despite treatment with rapid acting β2 agonist on a as needed basis and maintenance treatment with high dose inhaled glucocorticosteroid (ICS) plus long-acting β2 agonist plus 5 mcg tiotropium bromide mono hydrate; and
   (b) uncontrolled severe asthma in an adult patient whose asthma is not adequately controlled despite treatment with rapid acting β2 agonist on a as needed basis and maintenance treatment with high dose inhaled glucocorticosteroid (ICS) plus long-acting β2 agonist plus 5 mcg tiotropium bromide mono hydrate.
kkkkkkk) Use of a phosphodiesterase 4 (PDE4) inhibitor selected from the group consisting of Compound A and a pharmaceutically acceptable salt of Compound A for the manufacture of a pharmaceutical composition according to for the treatment of partly controlled severe asthma in an adult patient whose asthma is not adequately controlled despite treatment with rapid acting β2 agonist on a as needed basis and maintenance treatment with high dose inhaled glucocorticosteroid (ICS) plus long-acting β2 agonist plus 5 mcg tiotropium bromide mono hydrate.
lllllll) Use of a phosphodiesterase 4 (PDE4) inhibitor selected from the group consisting of Compound A and a pharmaceutically acceptable salt of Compound A for the manufacture of a pharmaceutical composition according to jjjjjjj) for the treatment of uncontrolled severe asthma in an adult patient whose asthma is not adequately controlled despite treatment with rapid acting β2 agonist on a as needed basis and maintenance treatment with high dose inhaled glucocorticosteroid (ICS) plus long-acting β2 agonist plus 5 mcg tiotropium bromide mono hydrate.
mmmmmmm) Use according to any one of jjjjjjj) to lllllll), wherein the phosphodiesterase 4 (PDE4) inhibitor is Compound A.
nnnnnnn) Use according to any one of jjjjjjj) to lllllll), wherein the phosphodiesterase 4 (PDE4) inhibitor is in an oral dosage form.
ooooooo) Use according to mmmmmmm), wherein the phosphodiesterase 4 (PDE4) inhibitor is in an oral dosage form.
ppppppp) Use according to nnnnnnn), wherein the oral dosage form includes an amount of Compound A selected from 0.150, 0.175, 0.2, 0.25, 0.3, 0.35, 0.4, 0.45, 0.5, 0.75 and 1.0 mg or an amount of a pharmaceutically acceptable salt of Compound A corresponding to 0.150, 0.175, 0.2, 0.25, 0.3, 0.35, 0.4, 0.45, 0.5, 0.75 or 1.0 mg of Compound A.
qqqqqqq) Use according to oooooo), wherein the oral dosage form includes an amount of Compound A selected from 0.150, 0.175, 0.2, 0.25, 0.3, 0.35, 0.4, 0.45, 0.5, 0.75 and 1.0 mg.
rrrrrrr) Use of a phosphodiesterase 4 (PDE4) inhibitor selected from the group consisting of Compound A and a pharmaceutically acceptable salt of Compound A and a leukotriene modifier selected from the group consisting of montelukast, pranlukast, zafirlukast, zileuton and a pharmaceutically acceptable salt of one of these compounds for the manufacture of a pharmaceutical composition for the treatment of any one of
   (a) partly controlled severe asthma in an adult patient whose asthma is not adequately controlled despite treatment with rapid acting β2 agonist on a as needed basis and maintenance treatment with high dose inhaled glucocorticosteroid (ICS) plus long-acting β2 agonist plus 5 mcg tiotropium bromide mono hydrate; and
   (b) uncontrolled severe asthma in an adult patient whose asthma is not adequately controlled despite treatment with rapid acting β2 agonist on a as needed basis and maintenance treatment with high dose inhaled glucocorticosteroid (ICS) plus long-acting β2 agonist plus 5 mcg tiotropium bromide mono hydrate.
sssssss) Use of a phosphodiesterase 4 (PDE4) inhibitor selected from the group consisting of Compound A and a pharmaceutically acceptable salt of Compound A and a leukotriene modifier selected from the group consisting of montelukast, pranlukast, zafirlukast, zileuton and a pharmaceutically acceptable salt of one of these compounds according to rrrrr) for the manufacture of a pharmaceutical composition for the treatment of partly controlled severe asthma in an adult patient whose asthma is not adequately controlled despite treatment with rapid acting β2 agonist on a as needed basis and maintenance treatment with high dose inhaled glucocorticosteroid (ICS) plus long-acting β2 agonist plus 5 mcg tiotropium bromide mono hydrate.
ttttttt) Use of a phosphodiesterase 4 (PDE4) inhibitor selected from the group consisting of

Compound A and a pharmaceutically acceptable salt of Compound A and a leukotriene modifier selected from the group consisting of montelukast, pranlukast, zafirlukast, zileuton and a pharmaceutically acceptable salt of one of these compounds according to rrrrr) for the manufacture of a pharmaceutical composition for the treatment of uncontrolled severe asthma in an adult patient whose asthma is not adequately controlled despite treatment with rapid acting β2 agonist on a as needed basis and maintenance treatment with high dose inhaled glucocorticosteroid (ICS) plus long-acting β2 agonist plus 5 mcg tiotropium bromide mono hydrate.
uuuuuuu) Use according to any one of rrrrrrr) to ttttttt), wherein the pharmaceutical composition comprises the phosphodiesterase 4 (PDE4) inhibitor and the leukotriene modifier in one single oral dosage form.
vvvvvvv) Use according to any one of rrrrrrr) to ttttttt), wherein the pharmaceutical composition comprises the phosphodiesterase 4 (PDE4) inhibitor and the leukotriene modifier in two separate oral dosage forms.
wwwwwww) Use according to any one of rrrrrrr) to ttttttt), wherein the pharmaceutical composition comprises the phosphodiesterase 4 (PDE4) inhibitor and the leukotriene modifier in two separate oral dosage forms and the two separate oral dosage forms are administered concurrently.
xxxxxxx) Use according to any one of rrrrrrr) to wwwwwww), wherein the phosphodiesterase 4 (PDE4) inhibitor is Compound A.
yyyyyyy) Use according to any one of rrrrrr) to wwwwwww), wherein the leukotriene modifier is montelukast or a pharmaceutically acceptable salt thereof.
zzzzzzz) Use according to xxxxxxx), wherein the leukotriene modifier is montelukast or a pharmaceutically acceptable salt thereof.
aaaaaaaa) Use according to uuuuuuu), wherein the one single oral dosage form includes an amount of Compound A selected from 0.150, 0.175, 0.2, 0.25, 0.3, 0.35, 0.4, 0.45, 0.5, 0.75 and 1.0 mg and an amount of montelukast sodium corresponding to 10 mg of montelukast
bbbbbbbb) Use according to wwwwwww), wherein
   (a) the oral dosage form comprising the phosphodiesterase 4 (PDE4) inhibitor contains Compound A in an amount selected from 0.150, 0.175, 0.2, 0.25, 0.3, 0.35, 0.4, 0.45, 0.5, 0.75 and 1.0 mg or an amount of a pharmaceutically acceptable salt of Compound A corresponding to 0.150, 0.175, 0.2, 0.25, 0.3, 0.35, 0.4, 0.45, 0.5, 0.75 or 1.0 mg of Compound A;
   (b) the oral dosage form comprising the leukotriene modifier contains montelukast sodium in an amount corresponding to 10 mg montelukast and
   (c) the pharmaceutical composition is administered once daily.

Various modifications of the invention, in addition to those described herein, will be apparent to those skilled in the art from the foregoing description. Such modifications are also intended to fall within the scope of the appended claims. Each reference, including, patents, patent applications and publications, cited in the present application is hereby incorporated by reference in its entirety. In the case of conflict or contradiction, the present disclosure controls.

1.-251. (canceled)

252. A method of treating severe asthma that is not adequately controlled despite prior treatment, comprising:
administering to a patient suffering from severe asthma that is not adequately controlled by prior treatment, a therapeutically effective amount of a phosphodiesterase 4 (PDE4) inhibitor selected from the group consisting of 5-((2R,4aR,10bR)-9-ethoxy-2-hydroxy-8-methoxy-1,2,3,4,4a,10b-hexahydro-phenanthridin-6-yl)-1-methylpyridin-2(1H)-one (hereinafter “Compound A”) and a pharmaceutically acceptable salt of Compound A.

253. The method according to claim 252, wherein the not adequately controlled severe asthma is partly controlled severe asthma, not adequately controlled
a) despite treatment according to GINA Step 4; or
b) despite treatment with a medium dose inhaled glucocorticosteroid (ICS) plus a long-acting β2-agonist; or
c) despite treatment with a rapid-acting β2-agonist on an as-needed basis and maintenance treatment with a medium dose inhaled glucocorticosteroid (ICS) plus a long-acting β2-agonist; or
d) despite treatment with a high dose inhaled glucocorticosteroid (ICS) plus a long-acting β2-agonist; or
e) despite treatment with a rapid-acting β2-agonist on an as-needed basis and maintenance treatment with a high dose inhaled glucocorticosteroid (ICS) plus a long-acting β2-agonist.

254. The method according to claim 252, wherein the not adequately controlled severe asthma is uncontrolled severe asthma, not adequately controlled
a) despite treatment according to GINA Step 4; or
b) despite treatment with a medium dose inhaled glucocorticosteroid (ICS) plus a long-acting β2-agonist; or
c) despite treatment with a rapid-acting β2-agonist on an as-needed basis and maintenance treatment with a medium dose inhaled glucocorticosteroid (ICS) plus a long-acting β2-agonist; or
d) despite treatment with a high dose inhaled glucocorticosteroid (ICS) plus a long-acting β2-agonist; or
e) despite treatment with a rapid-acting β2-agonist on an as-needed basis and maintenance treatment with a high dose inhaled glucocorticosteroid (ICS) plus a long-acting β2-agonist.

255. The method according to claim 253, wherein the patient is selected from one or more of: adult patient, adolescent patient 15 years of age and older and pediatric patient older than 5 years and younger than 15 years of age.

256. The method according to claim 254, wherein the patient is selected from one or more of: adult patient, adolescent patient 15 years of age and older and pediatric patient older than 5 years and younger than 15 years of age.

257. The method according to claim 252,
wherein the patient is also administered a leukotriene modifier selected from the group consisting of montelukast, and a pharmaceutically acceptable salt of montelukast such as montelukast sodium,
and wherein the phosphodiesterase 4 (PDE4) inhibitor and the leukotriene modifier are administered concurrently.

258. The method according to claim 257, wherein the concurrent administration is administered in one single dosage form.

259. The method according to claim 258, wherein the not adequately controlled severe asthma is partly controlled severe asthma, not adequately controlled
a) despite treatment according to GINA Step 4; or
b) despite treatment with a medium dose inhaled glucocorticosteroid (ICS) plus a long-acting β2-agonist; or
c) despite treatment with a rapid-acting β2-agonist on an as-needed basis and maintenance treatment with a medium dose inhaled glucocorticosteroid (ICS) plus a long-acting β2-agonist; or
d) despite treatment with a high dose inhaled glucocorticosteroid (ICS) plus a long-acting β2-agonist; or
e) despite treatment with a rapid-acting β2-agonist on an as-needed basis and maintenance treatment with a high dose inhaled glucocorticosteroid (ICS) plus a long-acting β2-agonist.

260. The method according to claim 258, wherein the not adequately controlled asthma is uncontrolled severe asthma, not adequately controlled
a) despite treatment according to GINA Step 4; or
b) despite treatment with a medium dose inhaled glucocorticosteroid (ICS) plus a long-acting β2-agonist; or
c) despite treatment with a rapid-acting β2-agonist on an as-needed basis and maintenance treatment with a medium dose inhaled glucocorticosteroid (ICS) plus a long-acting β2-agonist; or
d) despite treatment with a high dose inhaled glucocorticosteroid (ICS) plus a long-acting β2-agonist; or
e) despite treatment with a rapid-acting β2-agonist on an as-needed basis and maintenance treatment with a high dose inhaled glucocorticosteroid (ICS) plus a long-acting β2-agonist.

261. The method according to claim 259, wherein the patient is selected from one or more of: adult patient, adolescent patient 15 years of age and older and pediatric patient older than 5 years and younger than 15 years of age.

262. The method according to claim 260, wherein the patient is selected from one or more of: adult patient, adolescent patient 15 years of age and older and pediatric patient older than 5 years and younger than 15 years of age.

263. A Pharmaceutical composition comprising a phosphodiesterase 4 (PDE4) inhibitor selected from the group consisting of Compound A and a pharmaceutically acceptable salt of Compound A and a leukotriene modifier selected from the group consisting of montelukast and a pharmaceutically acceptable salt of montelukast, wherein the phosphodiesterase 4 (PDE4) inhibitor and the leukotriene modifier are in one single dosage form.

264. The pharmaceutical composition of claim 263 wherein the PDE4 inhibitor is Compound A and the leukotriene modifier is montelukast sodium.

265. The method according to claim 252,
wherein the patient is 5 years of age or younger,
wherein the not adequately controlled severe asthma is partly controlled severe asthma, not adequately controlled
a) despite treatment with double low dose inhaled glucocorticosteroid (ICS); or
b) despite treatment with a rapid-acting β2-agonist on an as-needed basis and maintenance treatment with a double low dose inhaled glucocorticosteroid (ICS).

266. The method according to claim 252,
wherein the patient is 5 years of age or younger,
wherein the not adequately controlled severe asthma is uncontrolled severe asthma, not adequately controlled
a) despite treatment with double low dose inhaled glucocorticosteroid (ICS); or
b) despite treatment with a rapid-acting β2-agonist on an as-needed basis and maintenance treatment with a double low dose inhaled glucocorticosteroid (ICS).

267. The method according to claim 258,
wherein the patient is 5 years of age or younger,
wherein the not adequately controlled severe asthma is partly controlled severe asthma, not adequately controlled
a) despite treatment with double low dose inhaled glucocorticosteroid (ICS); or
b) despite treatment with a rapid-acting β2-agonist on an as-needed basis and maintenance treatment with a double low dose inhaled glucocorticosteroid (ICS).

268. The method according to claim 258,
wherein the patient is 5 years of age or younger,
wherein the not adequately controlled severe asthma is uncontrolled severe asthma, not adequately controlled
a) despite treatment with double low dose inhaled glucocorticosteroid (ICS); or
b) despite treatment with a rapid-acting β2-agonist on an as-needed basis and maintenance treatment with a double low dose inhaled glucocorticosteroid (ICS).

269. The method according to claim 257, wherein the patient is selected from one or more of: adult patient, adolescent patient 15 years of age and older, and pediatric patient older than 5 years and younger than 15 years of age.

270. The method according to claim 252, wherein the PDE4 inhibitor administered to the patient is Compound A.

271. The method according to claim 257, wherein the PDE4 inhibitor administered to the patient is Compound A, and the leukotriene modifier administered to the patient is montelukast sodium.
